SEQUENCE LISTING (3 sequences)

SEQ ID NO: 1 - DNA, 20 bp, Artificial sequence
  Chemically synthesized
gaccggaacgatctcgcgta

SEQ ID NO: 2 - DNA, 20 bp, Artificial sequence
  Chemically synthesized
cgtctttgagccgagttggg

SEQ ID NO: 3 - DNA, 20 bp, Artificial sequence
  Chemically synthesized
gatttccggtgaaggatggg

(19) United States
(12) Patent Application Publication
(10) Pub. No.: US 2019/0151289 A1
(43) Pub. Date: May 23, 2019

(54) Identification of Small Molecule Inhibitors of Jumonji AT-Rich Interactive Domain 1A (JARID1A) Histone Demethylase

(71) Applicant: YALE UNIVERSITY, New Haven, CT (US)

(72) Inventors: Qin Yan, Stratford, CT (US); Molly Gale, New Haven, CT (US); Denton Hoyer, West Haven, CT (US); Joyce Sayegh, Danbury, CT (US); Jian Cao, East Haven, CT (US); Peter Gareiss, Milford, CT (US)

(21) Appl. No.: 16/099,790

(22) PCT Filed: May 12, 2017
(86) PCT No.: PCT/US17/32309

§ 371 Date: Nov. 8, 2018

Related U.S. Application Data
(60) Provisional application No. 62/335,777, filed on May 13, 2016.

(51) Int. Cl. A61K 31/4196; A61K 31/4439; A61K 45/06; A61P 35/00

(52) U.S. Cl. CPC A61K 31/4196; A61P 35/00; A61K 45/06; A61K 31/4439

(57) ABSTRACT

The present invention includes novel inhibitors of JARID1A demethylase activity, and methods using the same.

### CROSS-REFERENCE TO RELATED APPLICATIONS

The present invention claims priority to U.S. Provisional Application No. 62/335,777, filed May 13, 2016, which is hereby incorporated by reference in its entirety herein.

### STATEMENT REGARDING FEDERALLY SPONSORED RESEARCH OR DEVELOPMENT

This invention was made with government support under CA187862, CA191548 and CA016359 awarded by National Institutes of Health, W81XWH-14-1-0308 awarded by United States Army Medical Research and Material Command, and under 1122492 awarded by the National Science Foundation. The government has certain rights in the invention.

### BACKGROUND OF THE INVENTION

Epigenetic regulators read, write, and erase post-translational modifications of nucleic acids in DNA and amino acids in histone tails. These marks, such as methyl or acetyl groups, are important in the processes of DNA repair and replication and can function to dynamically regulate gene expression (Kouzarides, 2007, Cell 128:693-705). For example, near transcriptional start sites, lysine acetylation and methylation of histone 3 lysine 4 (H3K4) are generally associated with active gene transcription (Bernstein et al., 2007, Cell 128:669-681). Deregulation of epigenetic regulators is associated with many human diseases including inflammatory disorders, metabolic disorders, neurological disorders, and cancer (Arrowsmith et al., 2012, Nat. Rev. Drug Discovery 11:384-400). Small molecule inhibitors targeting regulators of the epigenome are becoming increasingly sought after for research and clinical uses. In fact, several histone deacetylase inhibitors and DNA methyltransferase inhibitors were approved for therapeutic use, and many more epigenetic drugs are in clinical trials (Dawson and Kourzarides, 2012, Cell 150:12-27).

Lysine demethylase 5A (JARID1A; KDM5A) belongs to the KDM5 or JARID1 family of demethylases, which also includes KDM5B (PLU1/JARID1B), KDM5C (SMCX/JARID1C), and KDM5D (SMCY/JARID1D) (Blair et al., 2011, Enzymes in Cancer 4:1383-1404). All family members are Jumonji C (JmjC) domain-containing enzymes that demethylate di- and tri-methylated lysine 4 on histone H3 through a dioxygenase reaction requiring cofactors Fe(II) and α-ketoglutarate (α-KG) (Klose et al., 2007, Cell 128:889-900; Tsukada et al., 2006, Nature 439:811-816. KDM5 enzymes contain several conserved domains including Jumonji N, JmjC, AT-rich interactive domain (ARID), and two or three plant homeodomains (PHD). High similarity in domain organization and homology in amino acid sequence is observed between members of the pairs KDM5A/KDM5B and KDM5C/KDM5D. KDM5A is often classified as a transcriptional repressor as it removes methyl groups from H3K4me2 and H3K4me3, marks associated with active promoters (Santos-Rosa et al., 2002, Nature 419:407-411). It has been shown to interact with the Sin3 corepressor complex (van Oevelen et al., 2008, Mol. Cell. 32:359-370) and RBP-J, a repressor of Notch target genes (Liefke et al., 2010, Genes & Dev. 24:590-601). However, the mono-methylated product of its demethylation reaction is associated with active enhancers. Thus, KDM5A may act as an activator by enriching H3K4me1 at enhancer regions, similar to KDM5C (Outchkourov et al., 2013, Cell Rep. 3:1071-1079).

KDM5A was originally discovered as a binding partner for the retinoblastoma protein (pRB) (Fattaey et al., 1993, Oncogene 8:3149-3156) and subsequently shown to antagonize the function of pRB in differentiation and senescence control (Benevolenskaya et al., 2005, Mol. Cell 18:623-635; Lin et al., 2011, Proc. Natl. Acad. Sci. U.S.A. 108:13379-13386). It is amplified in breast cancer (Hou et al., 2012, Am. J. Transl. Res. 4:247-256) and overexpressed in multiple human cancer types, including lung (Teng et al., 2013, Cancer Res. 73:4711-4721; Wang et al., 2013, PloS One 8:e84735), gastric (Zeng et al., 2010, Gastroenterology 138:981-992; Jiping et al., 2013, J. Cell Biochem. 114:2666-2672) and hepatocellular (Liang et al., 2013, PloS One 8:e69784) cancers. KDM5A contributes to several key steps of cancer progression including tumorigenesis, metastasis and drug tolerance (Rasmussen and Staller, 2014, Epigenomics 6:277-286). Knockout of KDM5A significantly slowed tumorigenesis in three different genetically engineered mouse models of cancer (Lin et al., Proc. Natl. Acad. Sci. U.S.A. 108:13379-13386; Cao et al., 2014, Cell Reports 6:868-877). For instance, KDM5A knockout drastically increased lifespan in Rb1^{+/-} mice, which developed pituitary and thyroid tumors (Lin et al., Proc. Natl. Acad. Sci. U.S.A. 108:13379-13386). Similarly, KDM5A loss prolonged survival in mice with loss of multiple endocrine neoplasia type 1 (MEN1) in their pancreatic islet cells, which developed neuroendocrine tumors (Lin et al., Proc. Natl. Acad. Sci. U.S.A. 108:13379-13386). In the MMTV-Neu breast cancer mouse model, loss of KDM5A slowed tumorigenesis as well as metastasis to the lungs (Cao et al., 2014, Cell Reports 6:868-877). Similarly, KDM5A was found to be important for epithelial-mesenchymal transition and invasion of lung cancer cells (Teng et al., 2013, Cancer Res. 73:4711-4721; Wang et al., 2013, PloS One 8:e84735). Furthermore, KDM5A expression is implicated in drug resistance to targeted anti-cancer therapies in both lung (Sharma et al., 2010, Cell 141:69-80) and breast cancer (Hou et al., 2012, Am. J. Transl. Res. 4:247-256), as well as in resistance to a DNA alkylating agent in glioblastoma (Banelli et al., 2015, Cell cycle 14:3418-3429). While there are several compounds that can inhibit the demethylase activity of KDM5A (Sayegh et al., 2013, J. Biol. Chem. 288:9408-9417; Wang et al., 2013, Nat. Comm. 4:2034; Itoh et al., 2015, ACS Med. Chem. Lett. 6:665-670; Suzuki and Miyata, 2011, J. med. Chem. 54:8236-8250; Bavetsias et al., 2016, J. Med. Chem.), there are currently no specific inhibitors shown to target KDM5A without inhibiting other members of the KDM5 family.

There is a need in the art for novel small molecule inhibitors of KDM5A. These inhibitors would prove useful in treating diseases related to the overactivity and/or overexpression of KDM5A, such as cancers. The present invention addresses and meets these needs.

### BRIEF SUMMARY OF THE INVENTION

The present invention includes a pharmaceutical composition comprising a compound, or a salt or solvate thereof, selected from the group consisting of a compound of formulae (I)-(IV):

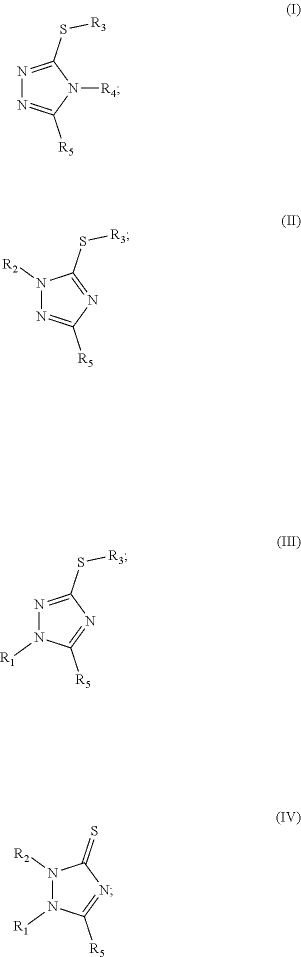

wherein in formulae (I)-(IV):

R¹, R², and R⁵ are each independently selected from the group consisting of H, C₁-C₆ alkyl, aryl-(C₁-C₃)alkyl, substituted aryl-(C₁-C₃)alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, and substituted heteroaryl;

R³ is selected from the group consisting of H, —C(O)R⁶, and —SR⁸;

R⁴ is selected from the group consisting of H, C₁-C₆ alkyl, substituted C₁-C₆ alkyl, C₂-C₆ alkenyl, aryl-(C₁-C₃)alkyl, substituted aryl-(C₁-C₃)alkyl, heteroaryl-(C₁-C₃)alkyl, substituted heteroaryl-(C₁-C₃)alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, and —NHR⁷;

R⁶ is selected from the group consisting of C₁-C₆ alkyl, aryl, and heteroaryl;

R⁷ is selected from the group consisting of H, C₁-C₆ alkyl, substituted C₁-C₆ alkyl, aryl, substituted aryl, aryl-(C₁-C₃)alkyl, substituted aryl-(C₁-C₃)alkyl, heteroaryl, substituted heteroaryl, heterocyclyl, substituted heterocyclyl, —C(O)R⁹, —S(O)R⁹, —S(O)₂R⁹;

R⁸ is selected from the group consisting of H, C₁-C₆ alkyl, substituted C₁-C₆ alkyl, C₂-C₆ alkenyl, C₃-C₇ cycloalkyl, substituted C₃-C₇ cycloalkyl, aryl, substituted aryl, heterocyclyl, substituted heterocyclyl, heteroaryl, and substituted heteroaryl; and

R⁹ is selected from the group consisting of C₁-C₆ alkyl, substituted C₁-C₆ alkyl, C₃-C₇ cycloalkyl, substituted C₃-C₇ cycloalkyl, aryl, substituted aryl, heterocyclyl, substituted heterocyclyl, heteroaryl, and substituted heteroaryl.

In one embodiment, the compound of formula (I) is a compound of formula (Ia), or a salt or solvate thereof:

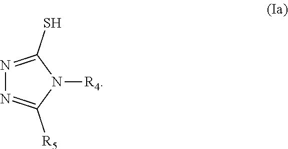

In one embodiment, the compound of formula (II) is a compound of formula (IIa), or a salt or solvate thereof:

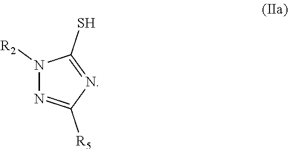

In one embodiment, the compound of formula (III) is a compound of formula (IIIa), or a salt or solvate thereof:

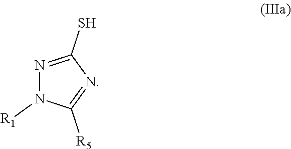

In one embodiment, the compound of formula (I) is a compound of formula (Ib), or a salt or solvate thereof:

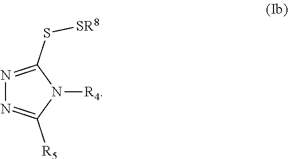

In one embodiment, R² is C₁-C₆ alkyl. In one embodiment, R³ is —C(O)R⁶, and R⁶ is selected from the group consisting of C₁-C₆ alkyl and aryl. In one embodiment, R⁴ is selected from the group consisting of H, C₁-C₆ alkyl, C₂-C₆ alkenyl, aryl, substituted aryl, heterocyclyl, aryl-(C₁-C₃)alkyl, heteroaryl-(C₁-C₃)alkyl, and —NHR⁷, wherein R⁷ is substituted aryl-(C₁-C₃)alkyl. In one embodiment, R⁵ is selected from the group consisting of H, C₁-C₆ alkyl, substituted C₁-C₆ alkyl, aryl, substituted aryl-(C₁-C₃)alkyl, substituted aryl, and heteroaryl. In one embodiment, the compound is selected from the group consisting of a compound of formula (Ia), a compound of formula (IIa), a compound of formula (IIIa), and a compound of formula (IV), or a salt or solvate thereof. In one embodiment, the compound is selected from the group consisting of:

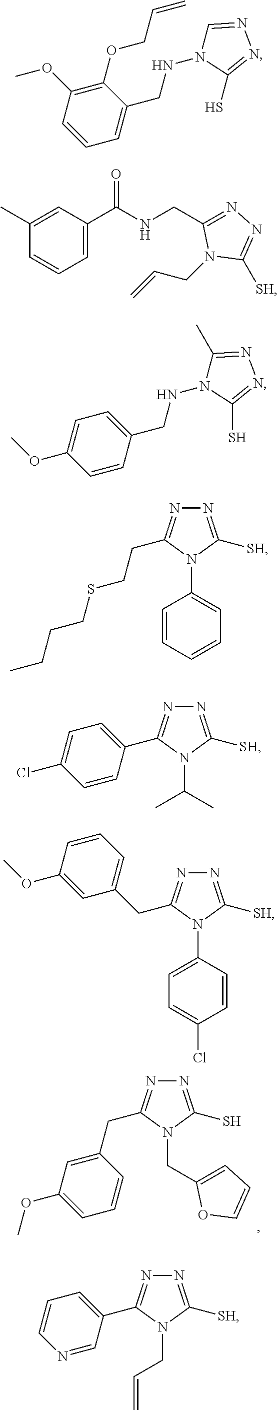

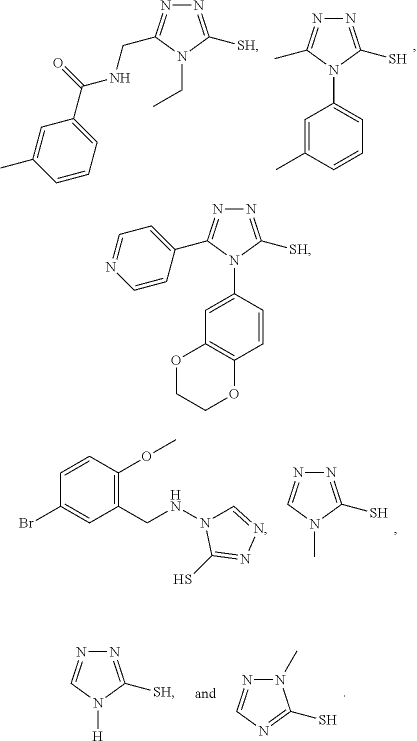

In one embodiment, the compound is selected from the group consisting of

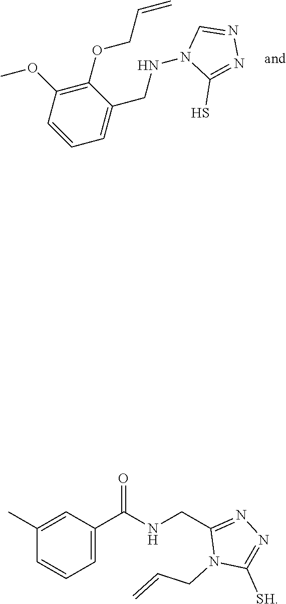 [SMILES: C=CCOc1c(CNn2cnnc2S)cccc1OC ; C=CCn1c(S)nnc1CNC(=O)c1cccc(C)c1]

The present invention also includes a method of treating or preventing cancer in a subject in need thereof. In one embodiment, the method comprising administering to the subject a therapeutically effective amount of a pharmaceutical composition comprising a compound selected from the group consisting of a compound of formulae (I)-(IV):

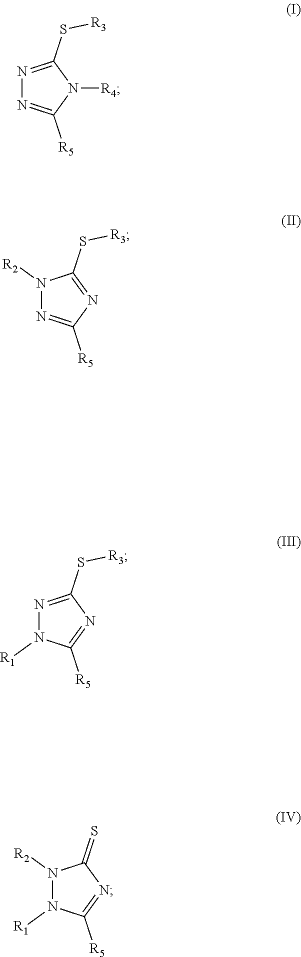

wherein in formulae (I)-(IV):

R¹, R², and R⁵ are each independently selected from the group consisting of H, C₁-C₆ alkyl, aryl-(C₁-C₃)alkyl, substituted aryl-(C₁-C₃)alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, and substituted heteroaryl;

R³ is selected from the group consisting of H, —C(O)R⁶, and —SR⁸;

R⁴ is selected from the group consisting of H, C₁-C₆ alkyl, substituted C₁-C₆ alkyl, C₂-C₆ alkenyl, aryl-(C₁-C₃)alkyl, substituted aryl-(C₁-C₃)alkyl, heteroaryl-(C₁-C₃)alkyl, substituted heteroaryl-(C₁-C₃)alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, and —NHR⁷;

R⁶ is selected from the group consisting of C₁-C₆ alkyl, aryl, and heteroaryl;

R⁷ is selected from the group consisting of H, C₁-C₆ alkyl, substituted C₁-C₆ alkyl, aryl, substituted aryl, aryl-(C₁-C₃)alkyl, substituted aryl-(C₁-C₃)alkyl, heteroaryl, substituted heteroaryl, heterocyclyl, substituted heterocyclyl, —C(O)R⁹, —S(O)R⁹, —S(O)₂R⁹;

R⁸ is selected from the group consisting of H, C₁-C₆ alkyl, substituted C₁-C₆ alkyl, C₂-C₆ alkenyl, C₃-C₇ cycloalkyl, substituted C₃-C₇ cycloalkyl, aryl, substituted aryl, heterocyclyl, substituted heterocyclyl, heteroaryl, and substituted heteroaryl; and

R⁹ is selected from the group consisting of C₁-C₆ alkyl, substituted C₁-C₆ alkyl, C₃-C₇ cycloalkyl, substituted C₃-C₇ cycloalkyl, aryl, substituted aryl, heterocyclyl, substituted heterocyclyl, heteroaryl, and substituted heteroaryl.

In one embodiment, the compound is selected from the group consisting of

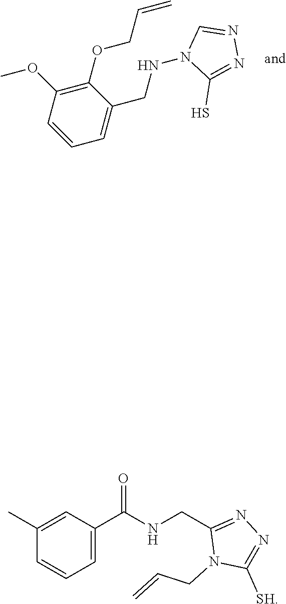 [SMILES: C=CCOc1c(CNn2cnnc2S)cccc1OC ; C=CCn1c(S)nnc1CNC(=O)c1cccc(C)c1]

In one embodiment, administration of the pharmaceutical composition to the subject inhibits the activity of at least one JARID1 demethylase in the subject. In one embodiment, the at least one JARID1 demethylase comprises JARID1A. In one embodiment, the cancer comprises a solid cancer. In one embodiment, the solid cancer is selected from the group consisting of breast cancer, prostate cancer, melanoma, lung cancer, gastric cancer, hepatocellular cancer, glioblastoma, neuroendocrine cancers, pancreatic cancer, and any combinations thereof and any combinations thereof. In one embodiment, the breast cancer comprises at least one HER2-positive breast cancer cell. In one embodiment, the at least one HER2-positive breast cancer cell is resistant to trastuzumab. In one embodiment, the lung cancer comprises at least one EGFR-mutant lung cancer cell. In one embodiment, the at least one EGFR-mutant lung cancer cell is resistant to gefitinib. In one embodiment, the subject is further administered an additional compound selected from the group consisting of a chemotherapeutic agent, an anti-cell proliferation agent, and any combinations thereof. In one embodiment, the chemotherapeutic agent comprises an alkylating agent, nitrosourea, antimetabolite, antitumor antibiotic, plant alkyloid, taxane, hormonal agent, bleomycin, hydroxyurea, L-asparaginase, or procarbazine. In one embodiment, the anti-cell proliferation agent comprises granzyme, a Bcl-2 family member, cytochrome C, or a caspase. In one embodiment, the pharmaceutical composition and the additional compound are co-administered to the subject. In one embodiment, the pharmaceutical composition and the additional compound are co-formulated and co-administered to the subject. In one embodiment, the pharmaceutical composition is administered to the subject by an administration route selected from the group consisting of inhalational, oral, rectal, vaginal, parenteral, topical, transdermal, pulmonary, intranasal, buccal, ophthalmic, intrathecal, and any combinations thereof. In one embodiment, the subject is a mammal. In one embodiment, the mammal is a human.

The present invention also includes a kit comprising an applicator, an instructional material for use thereof, and a compound selected from the group a compound selected from the group consisting of a compound of formulae (I)-(IV):

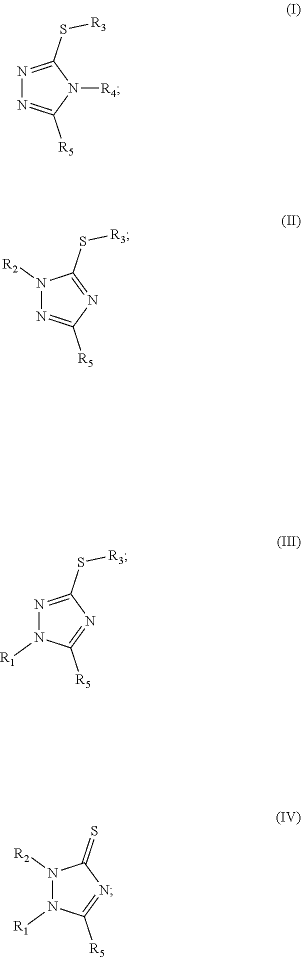

wherein in formulae (I)-(IV):

R¹, R², and R⁵ are each independently selected from the group consisting of H, C₁-C₆ alkyl, aryl-(C₁-C₃)alkyl, substituted aryl-(C₁-C₃)alkyl, substituted C₁-C₆ alkyl, C₁-C₆haloalkyl, C₃-C₇ cycloalkyl, substituted C₃-C₇ cycloalkyl, aryl, substituted aryl, heterocyclyl, substituted heterocyclyl, heteroaryl, and substituted heteroaryl;

R³ is selected from the group consisting of H, —C(O)R⁶, and —SR⁸;

R⁴ is selected from the group consisting of H, C₁-C₆ alkyl, substituted C₁-C₆ alkyl, C₂-C₆ alkenyl, aryl-(C₁-C₃)alkyl, substituted aryl-(C₁-C₃)alkyl, heteroaryl-(C₁-C₃)alkyl, substituted heteroaryl-(C₁-C₃)alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, and —NHR⁷;

R⁶ is selected from the group consisting of C₁-C₆ alkyl, aryl, and heteroaryl;

R⁷ is selected from the group consisting of H, C₁-C₆ alkyl, substituted C₁-C₆ alkyl, aryl, substituted aryl, aryl-(C₁-C₃)alkyl, substituted aryl-(C₁-C₃)alkyl, heteroaryl, substituted heteroaryl, heterocyclyl, substituted heterocyclyl, —C(O)R⁹, —S(O)R⁹, —S(O)₂R⁹;

R⁸ is selected from the group consisting of H, C₁-C₆ alkyl, substituted C₁-C₆ alkyl, C₂-C₆ alkenyl, C₃-C₇ cycloalkyl, substituted C₃-C₇ cycloalkyl, aryl, substituted aryl, heterocyclyl, substituted heterocyclyl, heteroaryl, and substituted heteroaryl; and

R⁹ is selected from the group consisting of C₁-C₆ alkyl, substituted C₁-C₆ alkyl, C₃-C₇ cycloalkyl, substituted C₃-C₇ cycloalkyl, aryl, substituted aryl, heterocyclyl, substituted heterocyclyl, heteroaryl, and substituted heteroaryl.

wherein the instructional material comprises instructions for preventing or treating cancer in a subject;

wherein the instructional material recites that the subject is administered a therapeutically effective amount of a pharmaceutical composition comprising the compound contained in the kit, whereby the cancer in the subject is treated or prevented.

In one embodiment, the cancer comprises breast cancer, prostate cancer, melanoma, lung cancer, gastric cancer, hepatocellular cancer, glioblastoma, and any combinations thereof. In one embodiment, the breast cancer comprises at least one HER2-positive breast cancer cell. In one embodiment, the at least one HER2-positive breast cancer cell is resistant to trastuzumab. In one embodiment, the lung cancer comprises at least one EGFR-mutant lung cancer cell. In one embodiment, the at least one EGFR-mutant lung cancer cell is resistant to gefitinib.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A-1J, depicts experimental data demonstrating the biochemical characterization of KDM5A using AlphaScreen. FIG. 1A is a schematic of the AlphaScreen assay used to measure demethylation of biotinylated H3K4me3 peptides by KDM5A. strep, streptavidin. FIG. 1B is an image of experimental western blots demonstrating the verification of affinity purified full-length FLAG-KDM5A by Coomassie Brilliant Blue stain (left) and anti-KDM5A western blot (right). MW, molecular weight; FT, flow-through. FIG. 1C is a graph of experimental data demonstrating the titration of FLAG-KDM5A in AlphaScreen assays. FIG. 1D is a graph of experimental data demonstrating the assessment of the specificity of the H3K4me1/2 antibody using mono-, di-, and tri-methylated H3K4 peptides. FIGS. 1E-1G are graphs of experimental data demonstrating the determination of the average apparent Kₘ of H3K4me3 peptide (FIG. 1E), α-KG (FIG. 1F), and Fe(II) (FIG. 1G), from two independent experiments. FIG. 1H is a graph of experimental data demonstrating the time course of the KDM5A demethylation reaction. FIGS. 1I-1J are graphs of experimental data demonstrating the titration of NaCl (FIG. 1I) and ZnCl₂ (FIG. 1J) in the KDM5A demethylation reaction. Data points in C-J represent mean±SD. Data are representative of at least two independent experiments performed in triplicate.

FIG. 2, comprising FIGS. 2A-2D, depicts experimental data demonstrating screen overview and top hits. FIG. 2A is a flow chart demonstrating the overview of the screening and hit selection process. FIG. 2B is a graph of experimental data demonstrating the validation of selected hits with a 3-thio-1,2,4-triazole core. The names and structures of these compounds are listed in Table 1. FIGS. 2C-2D are graphs of experimental data demonstrating the dose-response analysis for YUKA1 (FIG. 2C) and YUKA2 (FIG. 2D). Data are representative of at least four independent experiments performed in triplicate. Data points and bars in FIGS. 2B-2D indicate mean±SEM.

FIG. 3, comprising FIGS. 3A-3E, depicts experimental data demonstrating that YUKA1 and YUKA2 are KDM5A/C specific inhibitors. FIG. 3A is a table of experimental data demonstrating the average IC₅₀ values of YUKA1 and YUKA2 for members of the KDM5 and KDM6 families determined by at least three independent experiments performed in triplicate. FIGS. 3B-3E are graphs of experimental data demonstrating the activity of KDM5B (FIG. 3B), KDM5C (FIG. 3C), KDM6A (FIG. 3D) and KDM6B (FIG. 3E) with the indicated compounds in AlphaScreen assays. Bars in FIGS. 3B-3D indicate mean±SEM.

FIG. 4, comprising FIGS. 4A-4F, depicts experimental data demonstrating the mechanistic characterization of YUKA1 and YUKA2. FIGS. 4A-4B are graphs of experimental data demonstrating the dose-response analysis of YUKA1 (FIG. 4A) and YUKA2 (FIG. 4B) over a 16-fold range of concentrations of α-KG. FIGS. 4C-4D are graphs of experimental data demonstrating the inhibition of KDM5A by YUKA1 (FIG. 4C) and YUKA2 (FIG. 4D) in reactions with varying concentrations of Fe(II). FIGS. 4C-4D are graphs of experimental data demonstrating the dose-response analysis of YUKA1 (FIG. 4E) and YUKA2 (FIG. 4F) at the indicated Fe(II) concentrations. Data points in FIGS. 4A-4F indicate mean±SEM. Data are representative of at least two independent experiments performed in triplicate.

FIG. 5, comprising FIGS. 5A-5E, depicts experimental data demonstrating that YUKA1 is cell-active and selectively inhibits proliferation of KDM5A-dependent cancer cells. FIG. 5A is an image of a representative western blot analysis of H3K4me3 in HeLa (left) and MCF7 (right) cells after 48 hour treatment with the indicated compounds. Fold represents the relative ratio of band intensity for H3K4me3 divided by Total H3 loading control, normalized to DMSO control. FIG. 5B is series of graphs of experimental data of WST-1 proliferation assays of HeLa (left) and MCF7 (right) cells in the presence of YUKA1 and YUKA2 at the indicated concentrations. Bars indicate mean±SD of three independent experiments performed in quadruplicate. Asterisks indicate significance by unpaired t test (****, p<0.0001). D3, day 3; D0, day 0. FIG. 5C is a series of images of colony formation assays of HeLa (left) and MCF7 (right) cells treated with DMSO or YUKA1 for 12 days. Representative wells are shown. Quantification is shown in FIG. 7A. FIG. 5D is a series of images of representative western blot analyses of HeLa (left) and MCF7 (right) cells with doxycycline-induced KDM5A deletion using the CRISPR/Cas9 system. FIG. 5E is a series of images of colony formation assays of HeLa (left) and MCF7 (right) cells shown in panel D at 12 (HeLa) or 19 (MCF7) days after induction. Representative wells are shown. Quantification is shown in FIG. 7B. Dox, doxycycline; sg1, sgRNA 1; sg2, sgRNA 2.

FIG. 6, comprising FIGS. 6A-6D, depicts experimental data demonstrating the effect of YUKA1 on H3K4 methylation and proliferation of normal-like and cancer cells. FIG. 6A Representative western blot analysis of H3K4 methylation in ZR-75-1, HeLa, and MCF7 cells after 48 hr treatment with YUKA1, as well as MDA-MB-231 and MCF10A cells after 72 hr treatment with YUKA1. Total H3 serves as the loading control. Two exposures (light and dark) are shown for each blot. FIG. 6B is a graph of experimental data of WST-1 proliferation assays of ZR-75-1 cells treated with YUKA1 for 5 days. Bars indicate mean±SEM of four independent experiments performed in quadruplicate. FIG. 6C is a graph of experimental data of WST-1 proliferation assays of MDA-MB-231 cells treated with YUKA1 for 5 days. Bars indicate mean±SEM of three independent experiments performed in quadruplicate. FIG. 6D is a graph of experimental data of WST-1 proliferation assays of MCF10A cells treated with YUKA1 for 5 days. Bars indicate mean±SEM of three independent experiments performed in quadruplicate. In FIGS. 5B-5D, asterisks indicate significance by unpaired t test (*, p=0.03; ****, p<0.0001). D5, day 5

FIG. 7, comprising FIGS. 7A-7B, depicts experimental data demonstrating the quantification of colony formation assays shown in FIG. 5. FIG. 7A is a series of graphs of experimental data demonstrating the quantification of HeLa (left) and MCF7 (right) colony formation assays represented in FIG. 5C. Bars indicate mean±SD for two independent experiments in triplicate. Asterisks indicate significance by unpaired t test (****, p<0.0001). FIG. 7B is a series of graphs of experimental data demonstrating the quantification of HeLa (left) and MCF7 (right) cells represented in FIG. 5E. Bars indicate mean±SD. dox, doxycycline. Asterisks indicate significance by unpaired t test (*, p=0.02; **, p=0.004; NS, p>0.05). Relative intensity in FIGS. 7A-7B is the measured intensity value divided by the average value of DMSO-treated wells.

FIG. 8, comprising FIGS. 8A-8B, depicts experimental data demonstrating the effect of YUKA1 on anti-cancer drug resistance. FIG. 8A depicts images of colony formation assays of PC9 cells treated with DMSO or YUKA1 for 7 days (top wells), or treated with 2 μM gefitinib plus DMSO or YUKA1 for 35 days (bottom wells). FIG. 8B depicts images of colony formation assays of BT474 cells treated with DMSO or YUKA1 for 35 days (top wells), or treated with 5 μg/mL trastuzumab plus DMSO or YUKA1 for 42 days (bottom wells). Representative wells are shown in the top panel and quantification from three independent experiments performed in triplicate is shown in the bottom panel. Asterisks indicate significance by unpaired t test (***, p=0.0002; ****, p<0.0001). Bars indicate mean±SD. Relative intensity is the measured intensity value divided by the average value of DMSO-treated wells.

FIG. 9 is an image of representative Western blots demonstrating the expression of KDM5 demethylases in cell lines. Representative Western blot analysis of KDM5A (two different antibodies), KDM5B, and KDM5C in lung cancer cells (PC9), cervical cancer cells (HeLa), immortalized mammary epithelial cells (MCF10A), and breast cancer cell lines (MCF7, ZR-75-1, BT474, MDA-MB-231). Two exposures (light and dark) are shown for KDM5 blots. As expression of housekeeping loading control proteins varies across cell lines of different tissue types, equal loading was assessed by α-tubulin, GAPDH, vinculin, as well as Ponceau S membrane staining.

FIG. 10, comprising FIGS. 10A-10B, depicts experimental data demonstrating that catalase does not obstruct inhibition by YUKA1 and YUKA2. FIGS. 10A and 10B are graphs of experimental data demonstrating AlphaScreen assays assessing KDM5A activity performed in the presence of 0.01 mg/ml catalase or vehicle control reactions for YUKA1 (FIG. 10A) and YUKA2 (FIG. 10B). Data points and bars in A-B indicate mean±SD.

FIG. 11, comprising FIGS. 11A-11D, depicts experimental data demonstrating that YUKA1 (KDM5Ai), but not KDM5-C70 (pan-KDM5i), suppresses growth of HeLa and MDA-MB231 triple negative breast cancer cells. FIG. 11A is a series of images depicting colony-formation assays of cancer cell lines in HeLa cells. FIG. 11B is a series of images depicting colony-formation assays of cancer cell lines in MDA-MB231 cells. For FIGS. 11A and 11B, the cells were treated with DMSO, 5 μM KDM5-C70 or 50 or 100 μM YUKA1. FIGS. 11C and 11D are tables depicting the quantification of experimental data. “Ratio of % intensity” indicates the measured percent intensity normalized to the average percent intensity for DMSO-treated cells. Error bars denote SEM. **, p<0.01, ns, not significant.

### DETAILED DESCRIPTION OF THE INVENTION

The invention relates to the unexpected discovery of small molecule inhibitors of full length JARID1A (KDM5A) using a high-throughput screen using the AlphaScreen platform. The compounds of the invention showed great specificity for JARID1A and did not inhibit its close homologue JARID1B (KDM5B), nor the related H3K27 demethylases KDM6A (UTX) and KDM6B (JMJD3). Thus, the present invention also includes compounds of the present invention useful for the treatment of cancer.

JARID1A contributes to several key steps of cancer progression including tumorigenesis, metastasis and drug tolerance. Thus, the invention also relates to pharmaceutical compositions and methods of treating or preventing cancer in a subject using a compound of the invention. In one embodiment, the method comprises administering to the subject a therapeutically effective amount of a pharmaceutical composition comprising a compound of the invention. In one embodiment, the cancer comprises a solid cancer. In another embodiment, the solid cancer is selected from the group consisting of breast cancer, prostate cancer, melanoma, lung cancer, gastric cancer, hepatocellular cancer, glioblastoma, neuroendocrine cancers, pancreatic cancer, and any combinations thereof. In one embodiment, the subject is further administered an additional compound selected from the group consisting of a chemotherapeutic agent, an anti-cell proliferation agent and any combination thereof.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science and organic chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “α-KG” refers to alpha-ketoglutarate, or a salt or solvate thereof.

As used herein, the term “bio” refers to biotin or biotinylated.

As used herein, the term “DMSO” refers to dimethyl sulfoxide.

As used herein, the term “EDTA,” refers to ethylenediamine tetraacetic acid, or a salt or solvate thereof.

As used herein, the term “EGF” refers to epidermal growth factor.

As used herein, the term “FBS” refers to fetal bovine serum.

As used herein, the term “H3K4me1” refers to monomethylated lysine 4 in histone H3.

As used herein, the term “H3K4me2” refers to dimethylated lysine 4 in histone H3.

As used herein, the term “H3K4me3” refers to trimethylated lysine 4 in histone H3.

As used herein, the term “H3K27me2” refers to dimethylated lysine 27 in histone H3.

As used herein, the term “HER2+” refers to HER2 positive.

As used herein, the term “IC₅₀” refers to half maximal inhibitory concentration.

As used herein, the term “JARID1” refers to Jumonji AT-Rich Interactive Domain 1.

As used herein, the terms “KDM5A,” “RBP2,” and “JARID1A” are used interchangeably and refer to lysine demethylase 5A.

As used herein, the terms “KDM5B,” “PLU1,” and “JARID1B” are used interchangeably and refer to lysine demethylase 5B.

As used herein, the term “KDM5” refers to Lysine Demethylase 5.

As used herein, the term “JmjC” refers to jumonji.

As used herein, the term “trastuzumab” refers to a monoclonal antibody that interferes with the HER2/neu receptor (tradenames Herclon, Herceptin) (Hudis, 2007, N. Engl. J. Med. 3577(1):39-51).

As used herein, the term “gefitinib” refers to N-(3-chloro-4-fluoro-phenyl)-7-methoxy-6-(3-morpholin-4-ylpropoxy)quinazolin-4-amine.

As used herein, a “solvate” of a molecule refers to a complex between the molecule and a finite number of solvent molecules. In one embodiment, the solvate is a solid isolated from solution by precipitation or crystallization. In another embodiment, the solvate is a hydrate.

As used herein, a “subject” may be a human or non-human mammal or a bird. Non-human mammals include, for example, livestock and pets, such as ovine, bovine, porcine, canine, feline and murine mammals. Preferably, the subject is human.

As used herein, the term “cancer” is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the term “non-cancer control sample” as relating to a subject's tissue refers to a sample from the same tissue type, obtained from the patient, wherein the sample is known or found not to be afflicted with cancer. For example, a non-cancer control sample for a subject's lung tissue refers to a lung tissue sample obtained from the subject, wherein the sample is known or found not to be afflicted with cancer. “Non-cancer control sample” for a subject's tissue also refers to a reference sample from the same tissue type, obtained from another subject, wherein the sample is known or found not to be afflicted with cancer. “Non-cancer control sample” for a subject's tissue also refers to a standardized set of data (such as, but not limited to, identity and levels of gene expression, protein levels, pathways activated or deactivated etc.), originally obtained from a sample of the same tissue type and thought or considered to be a representative depiction of the non-cancer status of that tissue.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, an “effective amount”, “therapeutically effective amount” or “pharmaceutically effective amount” of a compound is that amount of compound that is sufficient to provide a beneficial effect to the subject to which the compound is administered.

The terms “treat” “treating” and “treatment,” as used herein, means reducing the frequency or severity with which symptoms of a disease or condition are experienced by a subject by virtue of administering an agent or compound to the subject.

The term “prevent,” “preventing” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “pharmaceutical composition” refers to a mixture of at least one compound useful within the invention with a pharmaceutically acceptable carrier. The pharmaceutical composition facilitates administration of the compound to a subject.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

In one aspect, the terms “co-administered” and “co-administration” as relating to a subject refer to administering to the subject a compound useful within the invention, or salt thereof, along with a compound that may also treat any of the diseases contemplated within the invention. In one embodiment, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

By the term “specifically bind” or “specifically binds,” as used herein, is meant that a first molecule preferentially binds to a second molecule (e.g., a particular receptor or enzyme), but does not necessarily bind only to that second molecule.

The terms “inhibit” and “antagonize”, as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C₃-C₆)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkynyl,” employed alone or in combination with other terms, means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂≡CCH. The term “homopropargylic” refers to a group exemplified by —CH₂CH₂≡CCH. The term “substituted propargylic” refers to a group exemplified by —CR₂—C≡CR, wherein each occurrence of R is independently H, alkyl, substituted alkyl, alkenyl or substituted alkenyl, with the proviso that at least one R group is not hydrogen. The term “substituted homopropargylic” refers to a group exemplified by —CR₂CR₂—C≡CR, wherein each occurrence of R is independently H, alkyl, substituted alkyl, alkenyl or substituted alkenyl, with the proviso that at least one R group is not hydrogen.

As used herein, the term “substituted alkyl,” “substituted cycloalkyl,” “substituted alkenyl” or “substituted alkynyl” means alkyl, cycloalkyl, alkenyl or alkynyl, as defined above, substituted by one, two or three substituents selected from the group consisting of halogen, —OH, alkoxy, tetrahydro-2-H-pyranyl, —NH₂, —N(CH₃)₂, (1-methyl-imidazol-2-yl), pyridin-2-yl, pyridin-3-yl, pyridin-4-yl, —C(═O)OH, trifluoromethyl, —C≡N, —C(═O)O(C₁-C₄)alkyl, —C(═O)NH₂, —C(═O)NH(C₁-C₄)alkyl, —C(═O)N((C₁-C₄)alkyl)₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “aromatic” refers to a carbocycle or heterocycle with one or more polyunsaturated rings and having aromatic character, i.e., having (4n+2) delocalized π(pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkyl chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkyl chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. As used herein, where a substituent is an alkyl or alkoxy group, the carbon chain may be branched, straight or cyclic, with straight being preferred.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention relates to a high-throughput screen for inhibitors of the JARID1 family of demethylases. This screen allows for the rapid and reliable identification of inhibitors of JARID1 demethylase activity. In one embodiment, the JARID1 demethylase screen allows for the identification of potent and specific inhibitors of JARID1A (KDM5A). Furthermore, the inhibitors described herein were found to be specific for JARID1A over its closely related family member JARID1B.

Very robust high throughput screens using the AlphaScreen platform are disclosed herein and facilitate searching for novel small molecule inhibitors of the histone lysine demethylase JARID1A. In one embodiment, the high-throughput screen of the invention utilizes full length JARID1A. In another embodiment, the substrate for the assay comprises bio-H3K4me3.

Described herein is a high throughput screen of small molecule JARID1A inhibitors. From this screen, several 3-thio-1,2,4-triazole compounds that inhibited JARID1A with low μM in vitro IC₅₀ values were identified. Moreover, these compounds showed great specificity and did not inhibit its close homologue JARID1B, nor the related H3K27 demethylases KDM6A (UTX) and KDM6B (JMJD3). One compound, named YUKA1, was able to increase H3K4me3 levels in human cells and selectively inhibit the proliferation of cancer cells whose growth depends on JARID1A. As JARID1A was shown to mediate drug tolerance, the ability of YUKA1 to prevent drug tolerance in EGFR-mutant lung cancer cells treated with gefitinib and HER2+ breast cancer cells treated with trastuzumab. The compound hindered the emergence of drug-tolerant cells, highlighting the critical role of JARID1A demethylase activity in drug resistance. YUKA1 was also found to suppress growth of HeLa and MDA-MB231 triple negative breast cancer cells, whereas KDMS-C70 (pan-KDM5i) did not.

The compounds described herein may be useful for further study of the demethylase activity of JARID1A, as well as its contributions to cancer. 3-thio-1,2,4-triazole and its derivative were found to be very potent inhibitors against JARID1A.

### Compositions

The invention includes a pharmaceutical composition comprising a compound, or a salt or solvate thereof, selected from the group consisting of a compound of formulae (I)-(IV):

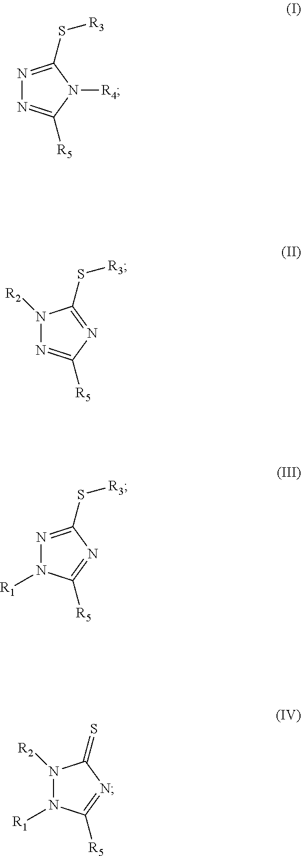

wherein in formulae (I)-(IV):

R¹, R², and R⁵ are each independently selected from the group consisting of H, C₁-C₆ alkyl, aryl-(C₁-C₃)alkyl, substituted aryl-(C₁-C₃)alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, and substituted heteroaryl;

R³ is selected from the group consisting of H, —C(O)R⁶, and —SR⁸;

R⁴ is selected from the group consisting of H, C₁-C₆ alkyl, substituted C₁-C₆ alkyl, C₂-C₆ alkenyl, aryl-(C₁-C₃)alkyl, substituted aryl-(C₁-C₃)alkyl, heteroaryl-(C₁-C₃)alkyl, substituted heteroaryl-(C₁-C₃)alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, and —NHR⁷;

R⁶ is selected from the group consisting of C₁-C₆ alkyl, aryl, and heteroaryl;

R⁷ is selected from the group consisting of H, C₁-C₆ alkyl, substituted C₁-C₆ alkyl, aryl, substituted aryl, aryl-(C₁-C₃)alkyl, substituted aryl-(C₁-C₃)alkyl, heteroaryl, substituted heteroaryl, heterocyclyl, substituted heterocyclyl, —C(O)R⁹, —S(O)R⁹, —S(O)₂R⁹;

R⁸ is selected from the group consisting of H, C₁-C₆ alkyl, substituted C₁-C₆ alkyl, C₂-C₆ alkenyl, C₃-C₇ cycloalkyl, substituted C₃-C₇ cycloalkyl, aryl, substituted aryl, heterocyclyl, substituted heterocyclyl, heteroaryl, and substituted heteroaryl; and

R⁹ is selected from the group consisting of C₁-C₆ alkyl, substituted C₁-C₆ alkyl, C₃-C₇ cycloalkyl, substituted C₃-C₇ cycloalkyl, aryl, substituted aryl, heterocyclyl, substituted heterocyclyl, heteroaryl, and substituted heteroaryl.

In one embodiment, the compound of formula (I) is a compound of formula (Ia), or a salt or solvate thereof:

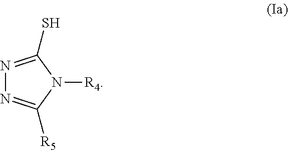

In one embodiment, the compound of formula (II) is a compound of formula (IIa), or a salt or solvate thereof:

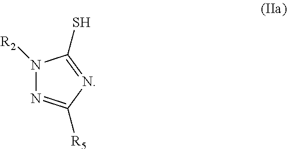

In one embodiment, the compound of formula (III) is a compound of formula (IIIa), or a salt or solvate thereof:

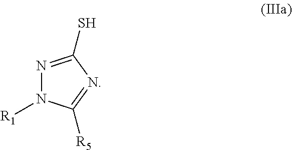

In one embodiment, the compound of formula (I) is a compound of formula (Ib), or a salt or solvate thereof:

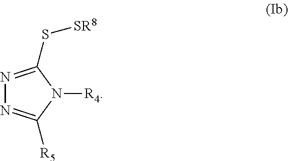

In one embodiment, R² is C₁-C₆ alkyl. In another embodiment, R² is alkyl. In one embodiment, R³ is H. In another embodiment, R³ is —C(O)R⁶. In one embodiment, R³ is —SR⁸. In one embodiment, R⁶ is selected from the group consisting of C₁-C₆ alkyl and aryl. In one embodiment, R⁶ is selected from the group consisting of methyl, ethyl, isopropyl, and phenyl. In one embodiment, R³ is selected from the group consisting of H, acetyl, and

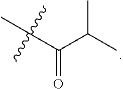 [SMILES: *C(=O)C(C)C]

In one embodiment, R⁴ is selected from the group consisting of H, C₁-C₆ alkyl, C₂-C₆ alkenyl, aryl, substituted aryl, heterocyclyl, aryl-(C₁-C₃)alkyl, heteroaryl-(C₁-C₃)alkyl, and —NHR⁷, wherein R⁷ is substituted aryl-(C₁-C₃)alkyl. In another embodiment, the substituted aryl is substituted with at least one substituent selected from the group consisting of Br, Cl, methoxy, allyloxy, and methyl. In another embodiment, R⁴ is selected from the group consisting of H, methyl, ethyl, isopropyl, phenyl, p-chlorophenyl, allyl, m-tolyl, —(CH₂)-furan, benzo-1,4-dioxyl,

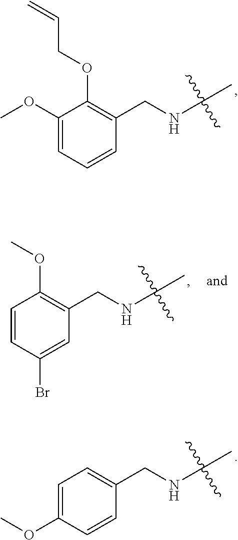 [SMILES: *NCc1cccc(OC)c1OCC=C ; *NCc1cc(Br)ccc1OC ; *NCc1ccc(OC)cc1]

In one embodiment, R⁵ is selected from the group consisting of H, C₁-C₆ alkyl, substituted C₁-C₆ alkyl, aryl, substituted aryl-(C₁-C₃)alkyl, substituted aryl, and heteroaryl. In another embodiment, the substituted C₁-C₆ alkyl is substituted with at least one substituent selected from the group consisting of —NHC(O)-substituted aryl and —S(C₁-C₆ alkyl). In another embodiment, the substituted aryl is substituted with at least one substituent selected from the group consisting of Cl, methyl, and methoxy. In another embodiment, R⁵ is selected from the group consisting of H, phenyl, p-chlorophenyl, 4-pyridyl, 3-pyridyl, 1-ethyl-3-methoxybenzyl, —(CH₂)₂—S—(CH₂)₃CH₃, and

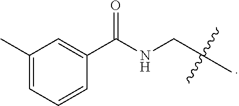 [SMILES: *CNC(=O)c1cccc(C)c1]

In one embodiment, the compound is selected from the group consisting of a compound of formula (Ia), a compound of formula (IIa), a compound of formula (IIIa), and a compound of formula (IV), or a salt or solvate thereof.

In one embodiment, the compound is selected from the group consisting of:

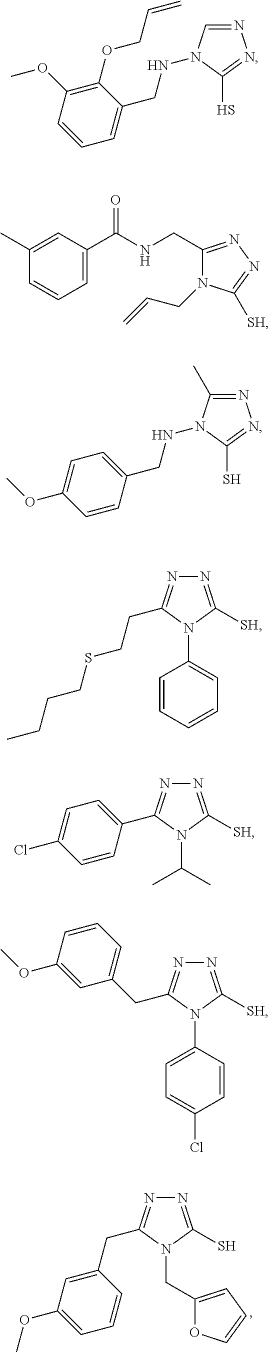

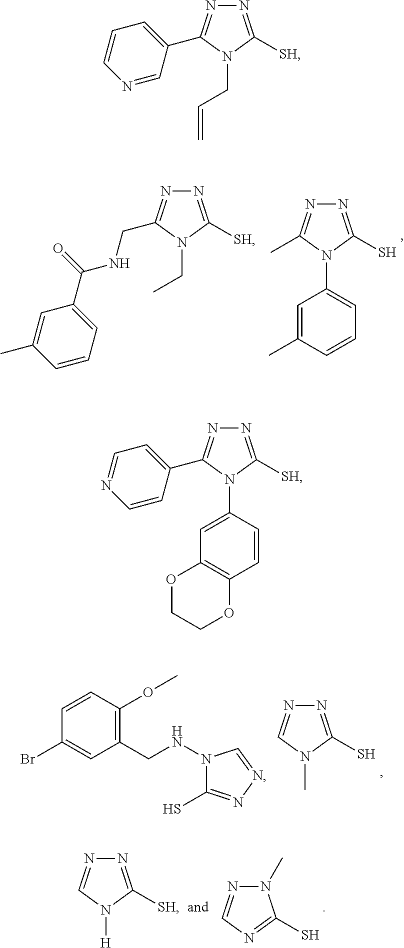

In one embodiment, the compound is selected from the group consisting of:

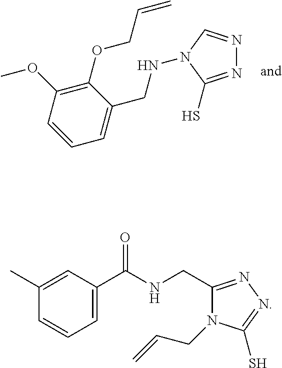 [SMILES: C=CCOc1c(CNn2cnnc2S)cccc1OC ; C=CCn1c(S)nnc1CNC(=O)c1cccc(C)c1]

The compounds useful within the invention may be prepared according to the general methodology known to those skilled in the art, or purchased from commercial suppliers as appropriate.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water, and ethanol. In another embodiment, the compounds described herein exist in unsolvated form.

Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomers is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/ or diastereomers. Resolution of compounds and isomers thereof is achieved by any means including, by way of non-limiting example, chemical processes, enzymatic processes, fractional crystallization, distillation, and chromatography.

In one embodiment, the compounds of the invention may exist as tautomers. All tautomers are included within the scope of the compounds presented herein.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically or therapeutically active form of the compound. In another embodiment, a prodrug is enzymatically or hydrolytically metabolized by one or more steps or processes to the biologically, pharmaceutically or therapeutically active form of the compound.

### Salts

The compounds described herein may form salts with acids, and such salts are included in the present invention. In one embodiment, the salts are pharmaceutically acceptable salts. The term “salts” embraces addition salts of free acids or bases that are useful within the methods of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine.

All of these salts may be prepared from the corresponding compound by reacting, for example, the appropriate acid or base with the compound.

### Combination Therapies

In one embodiment, the compounds of the invention are useful in the methods of present invention in combination with at least one additional compound useful for preventing and/or treating cancer. These additional compounds may comprise compounds of the present invention or other compounds, such as commercially available compounds, known to treat, prevent, or reduce the symptoms of cancer. In one embodiment, the combination of at least one compound of the invention or a salt thereof and at least one additional compound useful for preventing and/or treating cancer has additive, complementary or synergistic effects in the prevention and/or treatment of cancer.

In one aspect, the present invention contemplates that a compound useful within the invention may be used in combination with a therapeutic agent such as an anti-tumor agent, including but not limited to a chemotherapeutic agent, an anti-cell proliferation agent or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents.

Alkylating agents are so named because of their ability to add alkyl groups to many electronegative groups under conditions present in cells, thereby interfering with DNA replication to prevent cancer cells from reproducing. Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

In one embodiment, the therapeutic agent is a targeted cancer drugs. Non-limiting examples of targeted cancer drugs include trastuzumab, pertuzumab, gefitinib, erlotinib, bortezomib, and vemurafenib. In another embodiment, the therapeutic agent is an immune checkpoint inhibitor. Non-limiting examples of immune checkpoint inhibitors include keytruda, ipilimumab, and opdivo.

Anti-metabolites prevent incorporation of bases into DNA during the synthesis (S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include dactinomycin, daunorubicin, doxorubicin, idarubicin, mitomycin-C, and mitoxantrone.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine that are also useful in the invention.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In one embodiment, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In another embodiment, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof.

As used herein, combination of two or more compounds may refer to a composition wherein the individual compounds are physically mixed or wherein the individual compounds are physically separated. A combination therapy encompasses administering the components separately to produce the desired additive, complementary or synergistic effects. In one embodiment, the compound and the agent are physically mixed in the composition. In another embodiment, the compound and the agent are physically separated in the composition.

In one embodiment, the compound of the invention is co-administered with a compound that is used to treat cancer. The co-administered compound may be administered individually, or a combined composition as a mixture of solids and/or liquids in a solid, gel or liquid formulation or as a solution, according to methods known to those familiar with the art.

A synergistic effect may be calculated, for example, using suitable methods such as, for example, the Sigmoid-Eurax equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326), the median-effect equation (Chou & Talalay, 1984, Adv. Enzyme Regul. 22: 27-55), and through the use of isobolograms (Tallarida & Raffa, 1996, Life Sci. 58: 23-28). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Screening

The invention includes a high-throughput method of determining whether a compound inhibits JARID1A demethylase activity. The method comprises the step of providing tagged full length JARID1A enzyme. The method further comprises the step of incubating the tagged full length JARID1A enzyme with the compound and tagged H3K4Me3 peptide in a system at a determined temperature for a determined period of time. The method further comprises the step of determining whether any H3K4me2/1 peptide is formed in the system. If any H3K4me2/1 peptide is not formed in the system, the compound is determined to inhibit JARID1A demethylase activity.

In one embodiment, the tagged full length JARID1A enzyme comprises FLAG-tagged full length JARID1A enzyme. In another embodiment, the tagged H3K4Me3 peptide comprises biotinylated H3K4Me3 peptide. In yet another embodiment, the system further comprises alpha-ketoglutarate, an iron (II) salt and ascorbate. In yet another embodiment, determining whether any H3K4me2/1 peptide is formed in the system comprises incubating an H3K4me2 antibody or H3K4me1 antibody with at least a portion of the system. In yet another embodiment, the system is heterogeneous. In yet another embodiment, the tagged H3K4Me3 peptide is immobilized on a solid support.

### Methods

The invention includes a method of treating or preventing cancer in a subject. The method comprises administering to the subject a therapeutically effective amount of a pharmaceutical composition comprising a compound of the invention.

In one embodiment, administration of the pharmaceutical composition to the subject inhibits at least one JARID1 enzyme in the subject. In another embodiment, the at least one JARID1 enzyme comprises JARID1A. In another embodiment, the at least one JARID1 enzyme comprises JARID1A while not inhibiting JARID1B.

In one embodiment, the cancer comprises a solid cancer. In another embodiment, the solid cancer is selected from the group consisting of breast cancer, prostate cancer, melanoma, lung cancer, gastric cancer, hepatocellular cancer, glioblastoma, neuroendocrine cancers, pancreatic cancer, and any combinations thereof. In yet another embodiment, the breast cancer comprises HER2-positive breast cancer. In yet another embodiment, the HER2-positive breast cancer is resistant to trastuzumab. In yet another embodiment, the lung cancer comprises at least one EGFR-mutant lung cancer cell. In yet another embodiment, the at least one EGFR-mutant lung cancer cell is resistant to gefitinib.

In one embodiment, the subject is further administered an additional compound selected from the group consisting of a chemotherapeutic agent, an anti-cell proliferation agent and any combination thereof. In another embodiment, the chemotherapeutic agent comprises an alkylating agent, nitrosourea, antimetabolite, antitumor antibiotic, plant alkyloid, taxane, hormonal agent, bleomycin, hydroxyurea, L-asparaginase, or procarbazine. In yet another embodiment, the anti-cell proliferation agent comprises granzyme, a Bcl-2 family member, cytochrome C, or a caspase.

In one embodiment, the pharmaceutical composition and the additional compound are co-administered to the subject. In another embodiment, the pharmaceutical composition and the additional compound are co-formulated and co-administered to the subject. In yet another embodiment, the pharmaceutical composition is administered to the subject by an administration route selected from the group consisting of inhalational, oral, rectal, vaginal, parenteral, topical, transdermal, pulmonary, intranasal, buccal, ophthalmic, intrathecal, and any combinations thereof. In yet another embodiment, the subject is a mammal. In yet another embodiment, the mammal is a human.

### Kits

The invention includes a kit comprising an applicator, an instructional material for use thereof, and a compound of the invention.

The instructional material included in the kit comprises instructions for preventing or treating cancer in a subject. The instructional material recites that the subject is administered a therapeutically effective amount of a pharmaceutical composition comprising the compound contained in the kit. In one embodiment, the cancer comprises breast cancer, prostate cancer, melanoma, lung cancer, gastric cancer, hepatocellular cancer, glioblastoma, neuroendocrine cancers, pancreatic cancer, and any combinations thereof.

### Pharmaceutical Compositions and Formulations

The invention includes the use of pharmaceutical compositions of at least one compound of the invention or a salt thereof to practice the methods of the invention.

Such a pharmaceutical composition may consist of at least one compound of the invention or a salt thereof, in a form suitable for administration to a subject, or the pharmaceutical composition may comprise at least one compound of the invention or a salt thereof, and one or more pharmaceutically acceptable carriers, one or more additional ingredients, or some combination of these. The at least one compound of the invention may be present in the pharmaceutical composition in the form of a physiologically acceptable salt, such as in combination with a physiologically acceptable cation or anion, as is well known in the art.

In an embodiment, the pharmaceutical compositions useful for practicing the method of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the invention may be directly administered to the brain, the brainstem, or any other part of the central nervous system of a mammal or bird. Other contemplated formulations include projected nanoparticles, liposomal preparations, coated particles, resealed erythrocytes containing the active ingredient, and immunologically-based formulations. The route(s) of administration are readily apparent to the skilled artisan and depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions which are suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions of the invention are formulated using one or more pharmaceutically acceptable excipients or carriers. In one embodiment, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat cancer in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 mg/kg to 100 mg/kg of body weight/per day. One of ordinary skill in the art is able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of cancer in a patient.

In one embodiment, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physical taking all other factors about the patient into account.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments thereinbetween.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of cancer in a patient.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing cancer in a patient.

### Routes of Administration

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Injectable formulations may also be prepared, packaged, or sold in devices such as patient-contolled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (for example, see Constanza, U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. More preferable, it should be present in an amount from about 0.0005% to about 5% of the composition; most preferably, it should be present in an amount of from about 0.001% to about 1% of the composition. Such compounds may be synthetically-or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, that are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In a preferred embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 24 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 24 hours, about 12 hours, about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 24 hours, about 12 hours, about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

### Screen-Identified Selective Inhibitor of Lysine Demethylase 5A Blocks Cancer Cell Growth and Drug Resistance

The results described herein demonstrate the characterization of KDM5A using a homogeneous luminescence-based assay followed by a screen of about 9,000 small molecules. From this screen, several 3-thio-1,2,4-triazole compounds that inhibited KDM5A with low μM in vitro IC₅₀ values were identified. Importantly, these compounds showed great specificity and did not inhibit its close homologue KDM5B (PLU1/JARID1B) or the related H3K27 demethylases KDM6A (UTX) and KDM6B (JMJD3). One compound, named YUKA1, was able to increase H3K4me3 levels in human cells and selectively inhibit the proliferation of cancer cells whose growth depends on KDM5A. As KDM5A was shown to mediate drug tolerance, the ability of YUKA1 to prevent drug tolerance in EGFR-mutant lung cancer cells treated with gefitinib and HER2+ breast cancer cells treated with trastuzumab was investigated. YUKA1 was found to hinder the emergence of drug-tolerant cells, highlighting the critical role of KDM5A demethylase activity in drug resistance.

The materials and methods employed in these experiments are now described.

### Histone Peptides and Antibodies

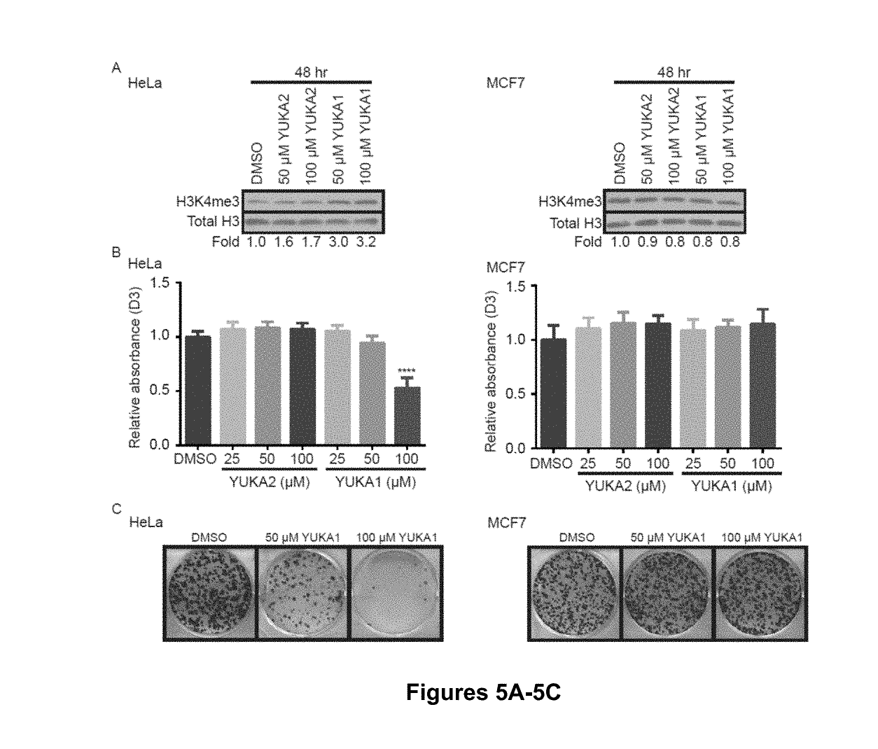

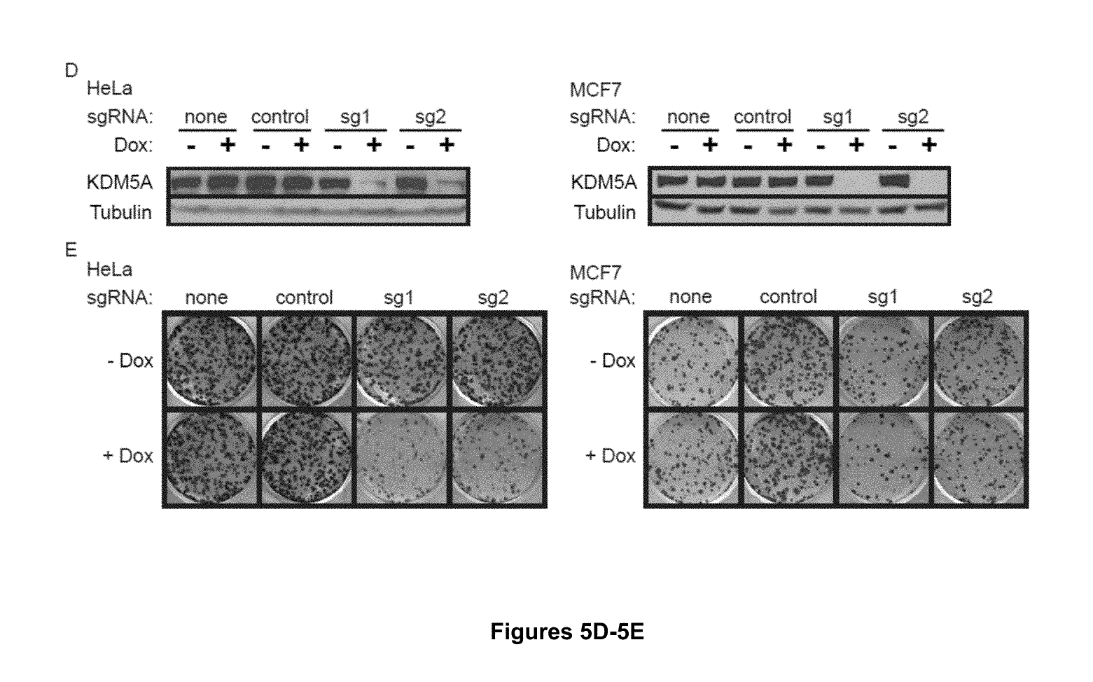

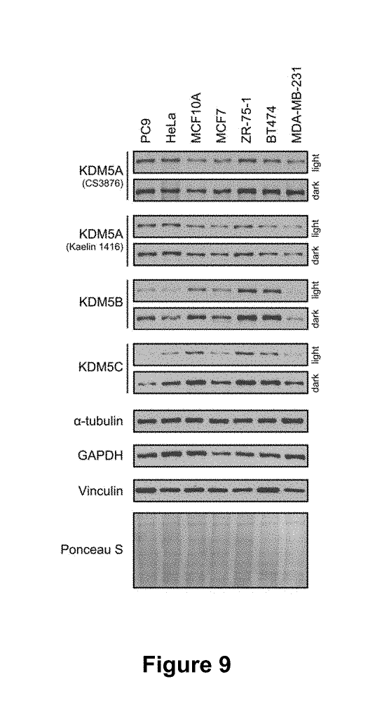

Biotinylated peptides were purchased from AnaSpec. Peptide sequences were described previously (Sayegh et al., 2013, J. Biol. Chem. 9408-9417). Anti-H3K4me1 (ab8895), anti-histone H3 (ab1791), and anti-GAPDH (ab9385) polyclonal antibodies were purchased from Abcam. Anti-H3K4me3 (CS9751), anti-H3K4me2 (CS9725) and anti-KDM5A (CS3876) monoclonal antibodies were purchased from Cell Signaling. Anti-H3K27me2 (07-452) polyclonal antibody was purchased from EMD Millipore. Anti-tubulin (T5168) and anti-vinculin (V9131) monoclonal as well as anti-KDM5B (HPA027179) polyclonal antibodies were purchased from Sigma. Anti-KDM5C polyclonal antibody (A301-035A) was purchased from Bethyl Laboratories, Inc. Anti-KDM5A antibody (Kaelin 1416) used in FIG. 5D and FIG. 9 was described previously (Klose et al., 2007, Cell 128:889-900).

### Cell Lines

Sf21 insect cells were cultured at 27° C. in Grace's medium (Gibco) with 10% fetal bovine serum (FBS) and 1% penicillin/streptomycin. HeLa cells were cultured in DMEM (Gibco) with 10% FBS and 1% penicillin/streptomycin. BT474, MCF7, MDA-MB-231, PC9 and ZR-75-1 cells were cultured in RPMI 1640 (Gibco) with 10% FBS and 1% penicillin/streptomycin. MCF10A cells were cultured as using previously described methods (Sayegh et al., 2013, J. Biol. Chem. 9408-9417). All human cells were cultured at 37° C. and 5% carbon dioxide. HeLa and MCF7 cell lines were authenticated using short tandem repeat profiling performed at the DNA Analysis Facility on Science Hill at Yale University. All other human cell lines used were within 10 passages after being obtained from the American Type Culture Collection.

### Enzyme Production

Sf21 insect cells were infected with baculovirus to express full-length FLAG-KDM5A (Klose et al., 2007, Cell 128:889-900). After three days at 27° C., cells were harvested and the enzyme was isolated using anti-FLAG M2 beads (Sigma). Samples were run on 7% SDS-PAGE gels, stained with Coomassie Brilliant Blue or used for western blot analysis to verify purity. FLAG-KDM5B, FLAG-KDM5C, and His-FLAG-KDM6B production was detailed previously (Sayegh et al., 2013, J. Biol. Chem. 9408-9417). FLAG-KDM6A was purchased from BPS Bioscience (50115).

### Western Blot Analysis

Cells were lysed using previously described methods (Lin et al., 2011, Proc. Natl. Acad. Sci. U.S.A. 108:13379-13386). Histones were separated by centrifugation, resuspended in Laemmeli buffer, and sonicated. Protein concentration of cell lysates was measured by Bradford assay. Samples in Laemmeli buffer were boiled 10 minutes at 95° C. and loaded onto 7% (whole cell lysates) or 18% (histones) SDS-PAGE gels. Membranes were blocked in 4% non-fat milk in Tris-buffered saline with 0.05% Tween (TBS-T) and incubated with primary antibodies in the same buffer or 5% bovine serum albumin in TBS-T overnight at 4° C. Membranes were incubated with secondary anti-rabbit or anti-mouse antibodies for one hour at room temperature. Blots were visualized by Thermo Scientific Pierce ECL Western Blotting Substrate (32106) or EMD Millipore Immobilon Western Chemiluminescent HRP Substrate (WBKLS0100) on film. Signal was quantified using ImageJ software.

### Chemicals

YUKA1 (7870547) and YUKA2 (7840569) were purchased from ChemBridge Hit2Lead. DMSO (9224-01) and sodium chloride (3624-07) were purchased from J. T. Baker. HEPES 1M buffer solution pH 7.3 (AB060201) and Coomassie Brilliant Blue G-250 (AB00325) were purchased from American BioAnalytical. L-ascorbic acid (4407-02) was purchased from Mallinckrodt Chemicals and 2-ketoglutaratic acid (K0005) was purchased from TCI America. Ferrous ammonium sulfate hexahydrate ACS reagent grade (152523) was purchased from MP Biomedicals. Zinc chloride (A16281) was purchased from Alfa Aesar. Trastuzumab (Herceptin) was purchased from Genentech. Gefitinib was purchased from Cayman Chemical Company (13166). Ponceau S was purchased from Acros (161470100).

### Demethylation Reactions

Histone demethylase assays were performed using previously described methods (Sayegh et al., 2013, J. Biol. Chem. 9408-9417), with the exception of all enzyme reactions containing 125 μM α-KG. Enzymes were used at the following approximate concentrations in validation experiments, chosen based on their activity in AlphaScreen assays: 19 nM FLAG-KDM5A, 15 nM FLAG-KDM5B, 6 nM FLAG-KDM5C, 29 nM His-FLAG-KDM6A, and 16 nM FLAG-KDM6B. 24 nM FLAG-KDM5A was used for Kₘ determination experiments. Reactions were carried out for 1 hour, or were stopped by addition of 30 mM EDTA every 5 minutes for determining enzyme kinetics. Kₘ values were calculated using the Michaelis-Menten non-linear regression analysis on GraphPad Prism 6.0 software. The concentration of DMSO in demethylation reactions was 0.05%. For reactions with catalase, catalase (Sigma C30) or an equal volume of dilution buffer was added to the peptide-containing buffer before addition of the demethylase. The final concentration of catalase in the reactions was 0.01 mg/ml.

### AlphaScreen Assay

The AlphaScreen general IgG (protein A) detection kit from PerkinElmer Life Sciences was used as described previously (Sayegh et al., 2013, J. Biol. Chem. 9408-9417). The luminescence emission was recorded at 570 nm using the AlphaScreen optic module on a Pherastar (BMG Labtech) or Envision (PerkinElmer Life Sciences) microplate reader.

### Screen

8,861 compounds from the ChemBridge MW-Set, ChemBridge DIVERSet, MicroSource Gen-Plus, MicroSource Pure Natural Products, NIH Clinical Collection, and Enzo Epigenetics libraries were screened for inhibition of FLAG-KDM5A demethylase activity. Compounds were dissolved in DMSO and added at 20 μM to 384-well white plates (Corning 3574) containing 64 nM bio-H3K4me3 peptide in demethylase buffer prior to addition of 13 nM FLAG-KDM5A. Hits were selected at a threshold of three standard deviations (˜30% inhibition). A counter-screen was performed with the hits to eliminate any non-specific compounds. For this, 20 μM compounds were added to 64 nM bio-H3K4me2 peptide in demethylase buffer with no enzyme. Any hits that interfered with the positive signal detection were eliminated. Dose-response curves were performed on selected hits using 0.1-11 μM of compound. Further validation of top hits was performed using 5 μM of each compound or a dose-response curve using 0.05-50 μM of the compound.

### Characterization of Top Compounds

Fresh compounds were ordered to confirm identity. IC₅₀ curves were generated for YUKA1 and YUKA2 using 0.05-50 μM compound in 1 hour reactions. IC₅₀ values were calculated by log transformation and non-linear regression log (inhibitor) versus response (three parameters) using Graphpad Prism 6.0 software. Percent activity of KDM5A was calculated for each data point by subtracting the background (average H3K4me3 signal) and dividing by the average signal for the DMSO controls. α-KG competition tests were performed using 150 nM H3K4me3 peptide and 50 μM Fe(II).

### Cell Proliferation Assay

WST-1 reagent from Roche Applied Sciences (11644807001) was used to measure cell number before drug addition and after 3 or 5 days of growth using previously described methods (Sayegh et al., 2013, J. Biol. Chem. 9408-9417). Cells were seeded in 96-well plates at the following number of cells per well: HeLa (2000), MCF7 (2000), ZR-75-1 (1000), MCF10A (1000), MDA-MB-231(5000). Assays were performed in technical quadruplicates and biological triplicates. DMSO was used at 0.1%. Relative absorbance was calculated by subtracting the average background (media only) signal, dividing by the average signal on day 0, and then dividing by the average day 5/day 0 ratio for the DMSO controls. Significance was calculated by unpaired, two-tailed student's t-test using GraphPad Prism 6.0 software.

### Knockout of KDM5A in Cell Lines

HeLa and MCF7 cells were infected with a lentiviral doxycycline-inducible Cas9-P2A-GFP construct. Cells highly expressing GFP after 2 days of 1 μg/ml doxycycline treatment were selected by flow cytometry and seeded one cell per well in a 96-well plate. Colonies with good GFP induction were harvested from this plate and infected with a lentiviral construct carrying sgRNA lacking a targeting sequence (none), control sgRNA (GACCGGAACGATCTCGCGTA (SEQ ID NO: 1)), or one of two sgRNAs targeting KDM5A (sg1: CGTCTTTGAGCCGAGTTGGG (SEQ ID NO: 2), sg2: GATTTCCGGTGAAGGATGGG (SEQ ID NO: 2)). Cells were selected by treatment with puromycin (1 μg/ml) for one week and continually cultured in puromycin afterwards. Knockout of KDM5A after a 3-day treatment with doxycycline (1 μg/ml) was confirmed by western blot.

### Colony Formation Assays

Cells were seeded in 6-well plates at low density: HeLa, MCF7, BT474, and PC9 cells at 1000, 2000, 2500, and 2000 cells/well respectively. Media containing the prescribed drugs or 0.1% DMSO was replaced every 3 days. Trastuzumab was used at 5 μg/ml. Gefitinib was used at 2 μM. YUKA1 was used at 50 or 100 μM. Doxycycline was used at 1 μg/ml for 3 days. After 7-42 days depending on the rate of cell growth, cells were fixed in 4% para-formaldehyde in phosphate-buffered saline for 10 minutes rocking at room temperature. They were incubated in 0.05% crystal violet in double-distilled water for 30 minutes rocking at room temperature, washed with water, and dried 24 hours before photographing. ColonyArea, a plugin for ImageJ software, was used for quantification (Guzman et al., 2014, PloS One 9:e92444). Unpaired, two-tailed student's t-test was used to determine significance using GraphPad Prism 6.0 software.

### Materials and Methods

The results of the experiments are now described.

### Biochemical Characterization of KDM5A

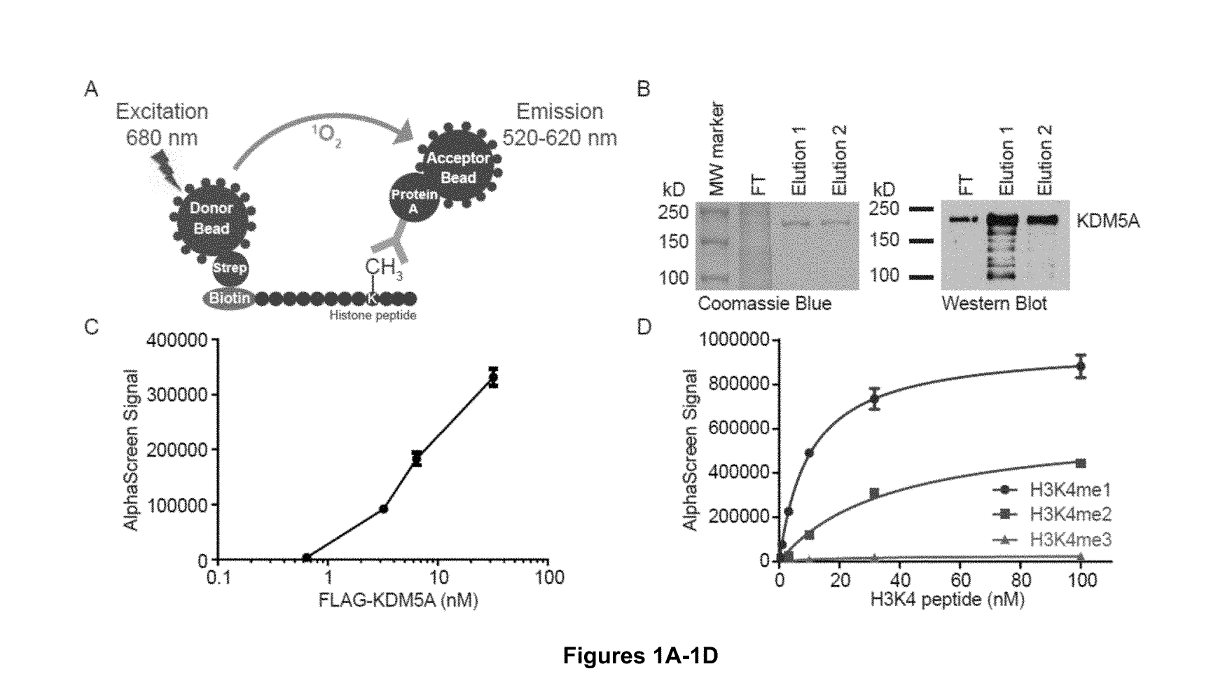

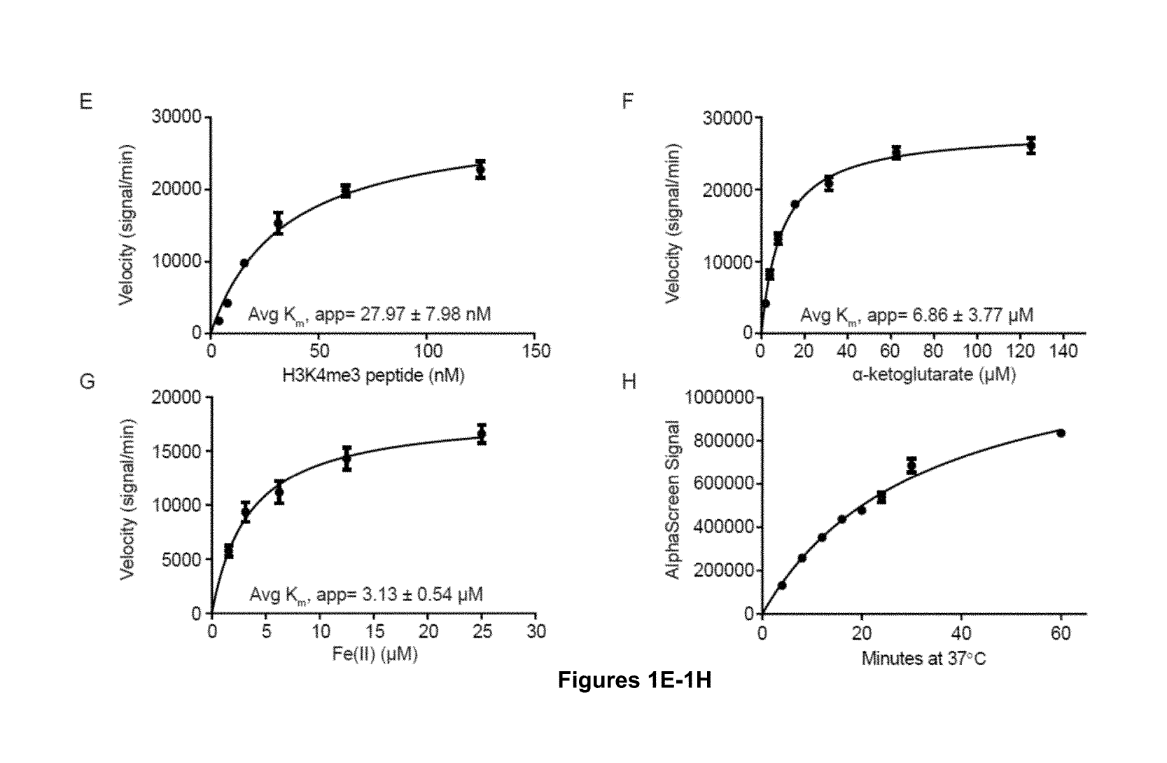

AlphaScreen technology (Perkin Elmer) was utilized to perform a screen for small molecule inhibitors of KDM5A. The assay was comprised of two steps, a demethylation reaction followed by detection of the product. A biotinylated H3K4me3 peptide was used as substrate in the demethylation reaction with KDM5A in the presence or absence of small molecule inhibitors. The presence of peptide product (H3K4me1/2) was detected using a product-specific antibody and beads. For this, acceptor beads coated in protein A bound to the antibody, which recognized the peptide product. Donor beads coated in streptavidin bound biotin on the peptide substrate. If the demethylation reaction occurred, the beads were in very close proximity and laser excitation of the donor beads at 680 nm caused a transfer of energy in the form of reactive singlet oxygen, resulting in emission by the acceptor beads between 520-620 nm (FIG. 1A) (Sakurai et al., 2010, Mol. Biosystems 6:357-364; Kawamura et al., 2010, Biochemistry 404:86-93. The luminescent signal detected was a proxy for the amount of demethylation that occurred.

FLAG-tagged full-length KDM5A was expressed in Sf21 insect cells and affinity purified using the FLAG tag. The purity of the isolated enzyme was assessed by SDS-PAGE and western blot (FIG. 1B). The enzyme showed strong activity by AlphaScreen even at low nM concentration (FIG. 1C). An antibody with an affinity for H3K4me1 that is about twice its affinity for H3K4me2 was selected, enabling detection of not only the incidence of demethylation, but the degree of demethylation (FIG. 1D). The affinity of the enzyme for the peptide in this assay was assessed by measuring the rate of the demethylation reaction over increasing peptide concentrations, leading to an average apparent Kₘ of about 28 nM (FIG. 1E). The average apparent Kₘ of α-KG was about 7 μM (FIG. 1F). Determination of the reaction rate over a range of Fe(II) concentrations revealed an average apparent Kₘ of about 3 μM (FIG. 1G). Under standard conditions, demethylation by FLAG-KDM5A increased linearly up to about 30 minutes, and continued to increase at a slower rate up to one hour (FIG. 1H). FLAG-KDM5A was sensitive to high salt concentrations, as the enzyme showed little activity with more than 50 mM NaCl (FIG. 1I). It was also sensitive to ZnCl₂ concentrations above 2 μM (FIG. 1J).

### Identification of Specific KDM5A Inhibitors by Screening

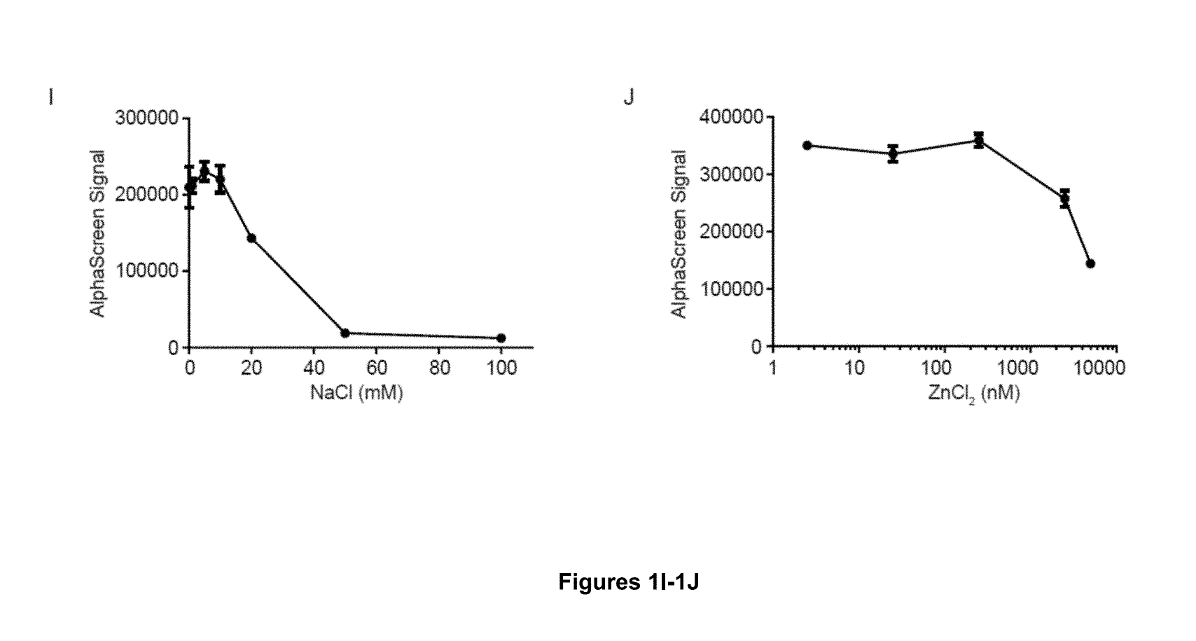

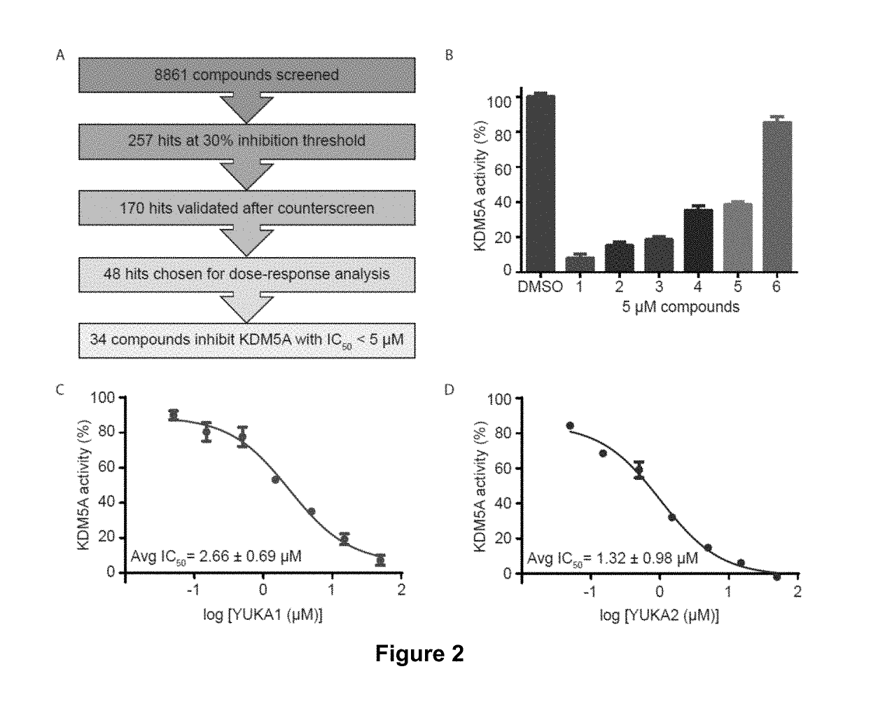

The screen (see schematic in FIG. 2A) included 8,861 compounds from selected small molecule libraries, including drugs approved for use in the clinic and diverse collections of compounds representing broad pharmacophore diversity and bioavailability. Screening statistics showed that the assay was sensitive and robust with a high average signal to background ratio (˜12) and an excellent average Z′ score (0.75). An inhibition threshold of three standard deviations (about 30% inhibition) identified 257 compounds. A counter-screen was used to eliminate any compounds that interfered with the assay itself by detecting the luminescent signal in the presence of the compound and positive control H3K4me2 peptide. The counter-screen validated 170 compounds. 48 compounds were chosen from this list for dose-response analysis, including 42 of the top 44 compounds with the highest potency at 20 μM and 6 compounds with drug-like structures. Among these 48 compounds, 34 compounds that inhibit KDM5A in vitro with half-maximal inhibitory concentrations (IC₅₀) of less than 5 μM.

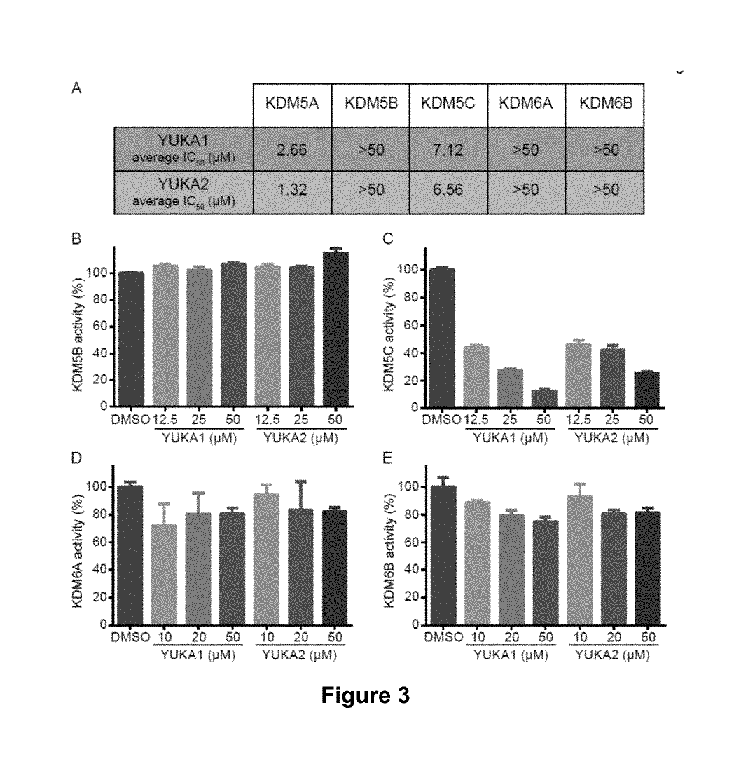

The screen identified several known JmjC demethylase inhibitors, as well as new inhibitor chemotypes. For instance, 2-4(4-methylphenyl)-1,2-benzisothiazol -3(2H)-one (PBIT), 2,4-pyridinedicarboxylic acid (2,4-PDCA), caffeic acid, and catechols like methyldopa, carbidopa and levodopa were among the active hits in the screen, validating the capability of our screening methods to identify inhibitors of KDM5A. Among the top hits, several 3-thio-1,2,4-triazole compounds were identified (Table 1, FIG. 2B). The two most potent inhibitors were focused on for further characterization: YUKA1 (4-([2-(allyloxy)-3-methoxybenzyl]amino)-4H-1,2,4-triazole-3-thiol) and YUKA2 (N-[(4-allyl-5-mercapto-4H-1,2,4-triazol-3-yl)methyl]-3-methylbenzamide), standing for Yale University KDM5A inhibitors 1 and 2. These inhibitors have average in vitro IC₅₀ values of 2.66 and 1.32 μM, respectively (FIGS. 2C, 2D). YUKA1 and YUKA2 showed no activity against KDM5A's close homologue KDM5B at 50 μM (FIGS. 3A, 3B) and are ˜3 and 5 fold less active against KDM5C, respectively (FIGS. 3A, 3C). Furthermore, 50 μM YUKA1 and YUKA2 did not half-maximally inhibit H3K27 demethylases KDM6A (UTX) and KDM6B (JMJD3) (FIGS. 3A, 3D, 3E). Therefore, YUKA1 and YUKA2 appear to be specific inhibitors of KDM5A and KDM5C.

TABLE 1
Selection of screen hits with a 3-thio-1,2,4-triazole core. Numbers
correspond to the compounds shown in FIG. 2.

| # | Name | Supplier ID | Structure |
|---|---|---|---|
| 1 | YUKA1 4-([2-(allyloxy)-3- methoxybenzyl]amino)- 4H-1,2,4-triazole- 3-thiol | ChemBridge 7870547 | 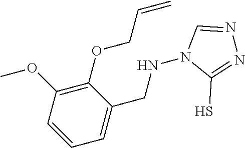 [SMILES: C=CCOc1c(CNn2cnnc2S)cccc1OC] |
| 2 | YUKA2 N-[(4-allyl-5-mercapto- 4H-1,2,4-triazol- 3-yl)methyl]-3- methylbenzamide | ChemBridge 7840569 | 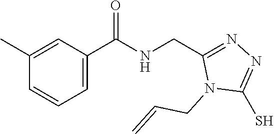 [SMILES: C=CCn1c(S)nnc1CNC(=O)c1cccc(C)c1] |
| 3 | 4-[(4- methoxybenzyl)amino]- 5-methyl-4H-1,2,4- triazole-3-thiol | ChemBridge 7985526 | 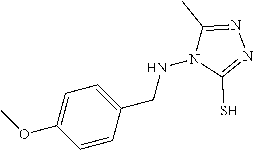 [SMILES: COc1ccc(CNn2c(C)nnc2S)cc1] |
| 4 | 5-[2-(butylthio)ethyl]-4- phenyl-4H- 1,2,4-triazole-3-thiol | ChemBridge 7918866 | 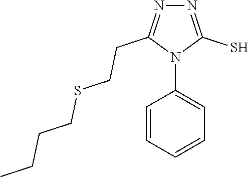 [SMILES: CCCCSCCc1nnc(S)n1-c1ccccc1] |
| 5 | 5-(4-chlorophenyl)-4- isopropyl-2,4-dihydro- 3H-1,2,4-triazole-3- thione | ChemBridge 7809088 | 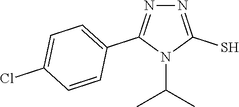 [SMILES: CC(C)n1c(S)nnc1-c1ccc(Cl)cc1] |
| 6 | 2-[(4-methyl-5-phenyl- 4H-1,2,4-triazol- 3-yl)thio]-1- phenylethanone | ChemBridge 7521464 | 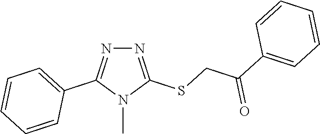 [SMILES: Cn1c(SCC(=O)c2ccccc2)nnc1-c1ccccc1] |

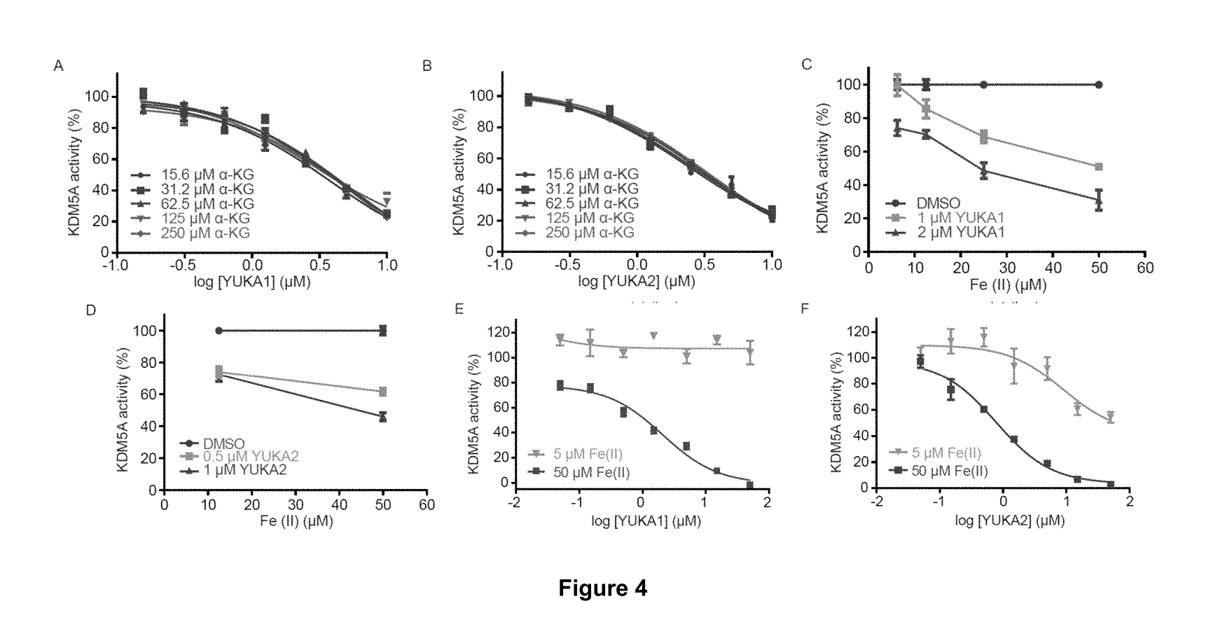

The specificity of these inhibitors prompted investigation of their mechanism of inhibition. Most characterized inhibitors of JmjC demethylases compete with cofactors required for the demethylase reaction, so competition analyses with these cofactors were performed. The IC₅₀ values for YUKA1 and YUKA2 did not change significantly over a wide range (16-fold) of concentrations of α-KG. Although not wishing to be bound by any particular theory, these results suggest that competition with α-KG is not the main mechanism of action (FIGS. 4A, 4B). These experiments were conducted using concentrations of peptide and Fe(II) several fold greater than their apparent Kₘ values in order to focus on the effect of α-KG alone. Analyzing the activity of FLAG-KDM5A at a range of Fe(II) concentrations revealed that Fe(II) is necessary for effective inhibition by YUKA1 and YUKA2 (FIGS. 4C, 4D). For example, inhibition of KDM5A activity by 2 μM YUKA1 ranged from ˜25% at low Fe(II) to ˜70% at 50 μM Fe(II). At low Fe(II) concentration (5 μM), enzyme inhibition was too weak to generate robust IC₅₀ curves for YUKA1 and YUKA2, as was done easily at 50 μM Fe(II) (FIGS. 4E, 4F). Although not wishing to be bound by any particular theory, these results suggest that YUKA1 and YUKA2 are uncompetitive with respect to Fe(II), requiring enzyme-bound Fe(II) for inhibition.

### KDM5A Inhibitor YUKA1 Inhibited Cancer Cell Proliferation and Drug Resistance

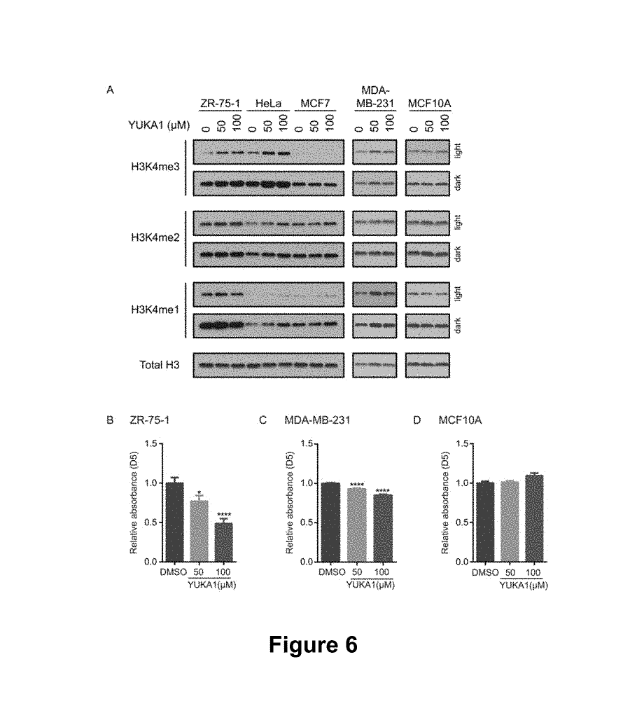

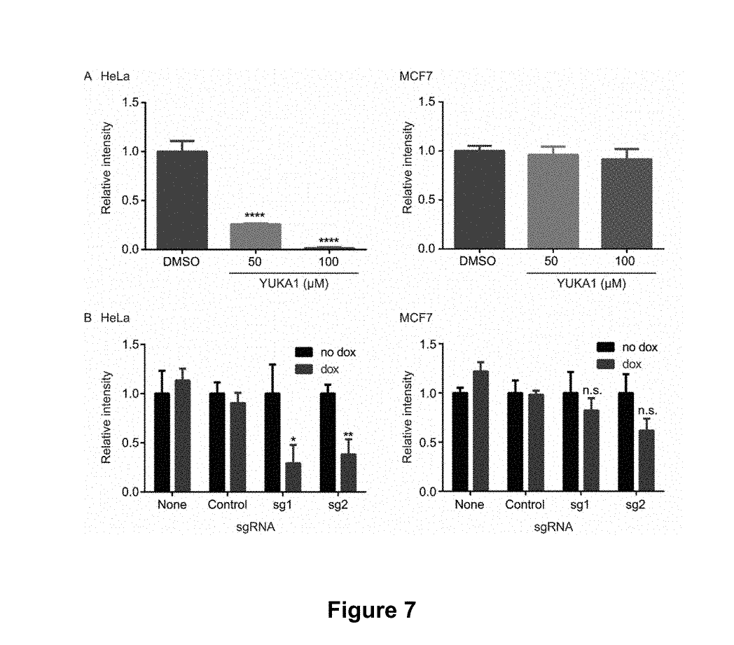

YUKA1 and YUKA2 were tested for their abilities to inhibit KDM5A in vivo using HeLa cervical cancer and MCF7 breast cancer cell lines. Western blot analysis of global H3K4 methylation changes revealed that YUKA1, but not YUKA2, was cell-active (FIG. 5A). These results are consistent with the fact that YUKA2 possesses a polar amide bond, which likely hinders its permeability across the cell membrane. A dose-dependent increase in global H3K4me3 levels was observed after 48 hour treatment with YUKA1 in HeLa cells, but not in MCF7 cells (FIG. 5A). H3K4me2 and H3K4me1 levels were also increased in YUKA1-treated HeLa cells, but not MCF7 cells (FIG. 6A). The ability of YUKA1 to change global H3K4 methylation levels correlated with its ability to affect the rate of cell proliferation. Proliferation of HeLa cells treated with YUKA1 was less than half of DMSO-treated cells after 3 days, while MCF7 cells were not affected (FIG. 5B). Likewise, the number of colonies formed by HeLa cells after a two-week treatment with YUKA1 was significantly reduced compared to treatment with DMSO control, but MCF7 cells treated with YUKA1 formed a similar number of colonies as the control (FIGS. 5C and 7A). To confirm the differential effects of KDM5A inhibition in these two cell lines, HeLa and MCF7 cells with doxycycline-inducible Cas9-mediated knockout of KDM5A were generated (FIG. 5D) and the effects of KDM5A loss on colony formation was examined. Consistent with YUKA1 inhibition, KDM5A loss significantly decreased the ability of HeLa cells to form colonies, but had little effect on MCF7 cells, as shown by comparing the doxycycline-treated wells to the untreated control wells (FIGS. 5E and 7B).

In order to further validate the cellular function of YUKA1, YUKA1 was tested in ZR-75-1 breast cancer cells, a cell line with KDM5A amplification and in which RNAi-mediated knockdown of KDM5A resulted in decreased cell proliferation (Hou et al., 2012, Am. J. Transl. Res. 4:247-256). It was observed that 48 hour treatment with YUKA1 increased global H3K4me3 levels in this cell line (FIG. 6A), as well as decreased cell proliferation during 5 days of treatment in a dose-dependent manner (FIG. 6B). The triple negative breast cancer cell line MDA-MB-231 showed only minor changes in H3K4 methylation, accompanied by a small decrease (10-15%) in cell proliferation during 5 days of treatment (FIGS. 6A and 6C). In comparison, treatment with YUKA1 did not affect H3K4 methylation levels and did not hinder cell proliferation of the normal-like MCF10A immortalized mammary epithelial cells (FIGS. 6A and 6D). This corresponds to previous data showing that knocking down KDM5A in MCF10A cells did not reduce cell growth (Hou et al., 2012, Am. J. Transl. Res. 4:247-256).

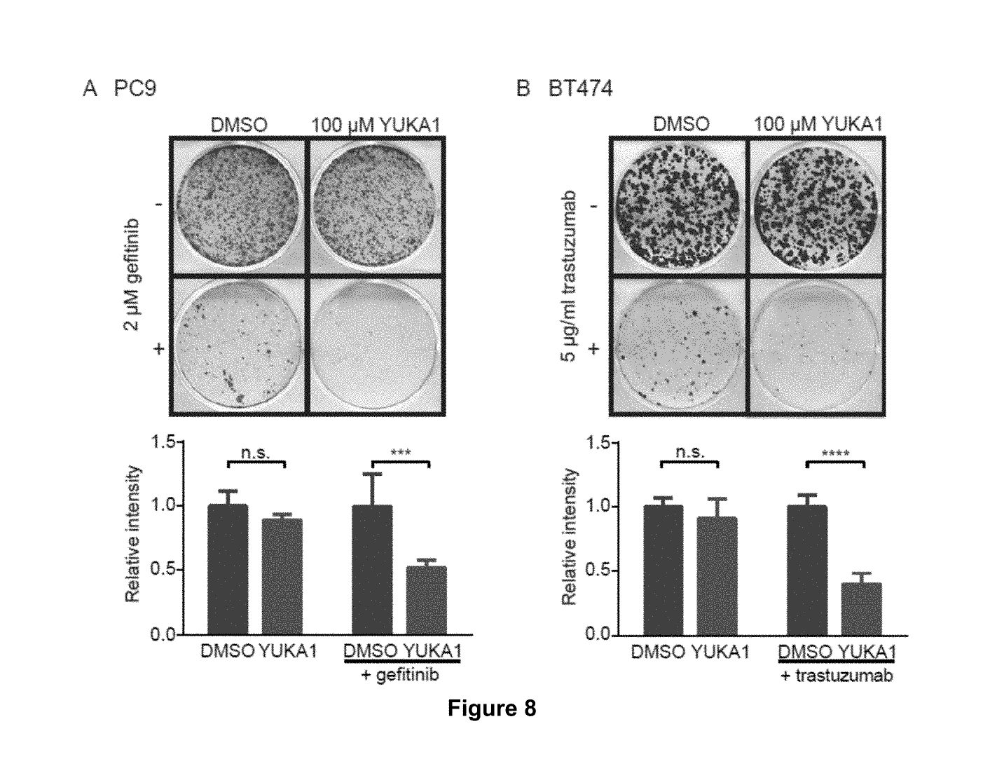

KDM5A was shown to be a powerful mediator of drug tolerance to gefitinib, a small molecule inhibitor of the epidermal growth factor receptor (EGFR), in the EGFR-mutant lung cancer cell line PC9 (Sharma et al., 2010, Cell 141:69-80). However, it was not known whether the demethylase activity of KDM5A actively contributed to this phenotype. Colony formation assays showed that fewer YUKA1-treated cells formed colonies during long-term treatment with 2 μM gefitinib compared to control cells treated with DMSO (FIG. 8A). Growth of PC9 cells was not significantly affected by treatment with YUKA1 alone, which corresponded to prior data showing that KDM5A knockdown did not affect short term proliferation of PC9 cells (Sharma et al., 2010, Cell 141:69-80). The effects of YUKA1 in a different setting of anti-cancer drug resistance was observed using human epidermal growth factor receptor-positive (HER2+) BT474 breast cancer cells treated with the monoclonal antibody trastuzumab (trade name Herceptin). BT474 cell growth was not changed when treated with YUKA1 alone, but emergence of colonies subjected to a low dose of trastuzumab (5 μg/ml) was significantly less for cells treated with YUKA1 (FIG. 8B). These experiments support the hypothesis that the demethylase activity of KDM5A is necessary for both lung and breast cancer cells to develop resistance to targeted therapies.

As described herein, a screen was performed using the full-length KDM5A protein, an approach which enables identification of new inhibitor chemotypes that may not be discovered by screening against truncated KDM5A or by structure-guided design. The screen was performed using the AlphaScreen platform, which is cost-effective, highly sensitive, and requires only small amounts of enzyme (Gale and Yan et al., 2015, Epigenomics 7:57-65).

Among ˜9,000 compounds screened, 34 compounds were identified that inhibited KDM5A with IC₅₀ values in the low μM range (<5 μM). Several compounds were previously shown as inhibitors of JmjC demethylases, validating the results from the screen. This screen revealed a novel inhibitor chemotype that includes a core structure of 3-thio-1,2,4-triazole (Table 1). Known inhibitors of KDM5A are pan-KDM5 demethylase inhibitors, with strong inhibitory effects on the other KDM5 family members or they were not evaluated for specificity within the KDM5 family. YUKA1 and YUKA2 were found to be potent and specific inhibitors of KDM5A. They showed little to no activity in biochemical assays against KDM5B, KDM6A and KDM6B even at the highest tested concentration of 50 μM and were less potent against KDM5C (FIG. 3). This specificity was also demonstrated in cell-based assays. HeLa and ZR-75-1 cells depend on KDM5A expression for their proliferation (FIG. 5E) (Hou et al., 2012, Am. J. Transl. Res. 4:247-256). The cell permeable compound YUKA1 increased H3K4me3 levels and inhibited growth of HeLa and ZR-75-1 cells (FIGS. 5 and 6). In contrast, MCF7 cells, which were shown to be sensitive to KDM5B down-regulation (Yamane et al., 2007, Mol. Cell 25:801-812; Yamamoto et al., 2014, Cancer Cell 25:762-777), were not significantly affected by KDM5A deletion or inhibition by YUKA1 (FIG. 5). Likewise, the normal-like MCF10A cells and PC9 lung cancer cells were not affected by KDM5A knockdown or by treatment with YUKA1 (FIGS. 6D and 8A) (Hou et al., 2012, Am. J. Transl. Res. 4:247-256; Sharma et al., 2010, Cell 141:69-80). To determine whether the expression levels of KDM5s correlate with YUKA1 sensitivity, the protein levels of KDM5A, B, and C were examined in all of the cell lines that were tested (FIG. 9). KDM5D was not examined because KDM5D is located on the Y chromosome and therefore is not expressed in the breast cancer cell lines derived from female patients. Though YUKA1-sensitive HeLa and ZR-75-1 cells expressed relatively higher levels of KDM5A compared to other cell lines, sensitivity to YUKA1 did not strictly correlate with expression levels of KDM5A, B, or C. Instead, sensitivity appears to correlate with the ability of YUKA1 to influence H3K4 methylation in the cell lines.

There are currently no chemical tools to study the demethylase activity of KDM5A separately from that of its family member KDM5B. YUKA1 and YUKA2 are unique in that they inhibit KDM5A with much greater potency than KDM5B. This feature of these compounds makes them useful tools for exploring of the biology of KDM5 enzymes. Importantly, YUKA1 is cell-active and can serve as a way to ascertain the significance of KDM5A's demethylase activity in cells.

YUKA1 and YUKA2 appear to inhibit KDM5A via a novel mechanism of action. As opposed to the α-KG analogues and iron chelators found to inhibit other JmjC demethylases, these inhibitors do not appear to compete with α-KG and require Fe(II) for effective inhibition (FIG. 4). Since thiols are known to have a high affinity for iron, it is hypothesized that these compounds bind iron at an open coordination site or possibly displace one or more of iron's natural ligands, thereby disrupting the catalytic cycle.

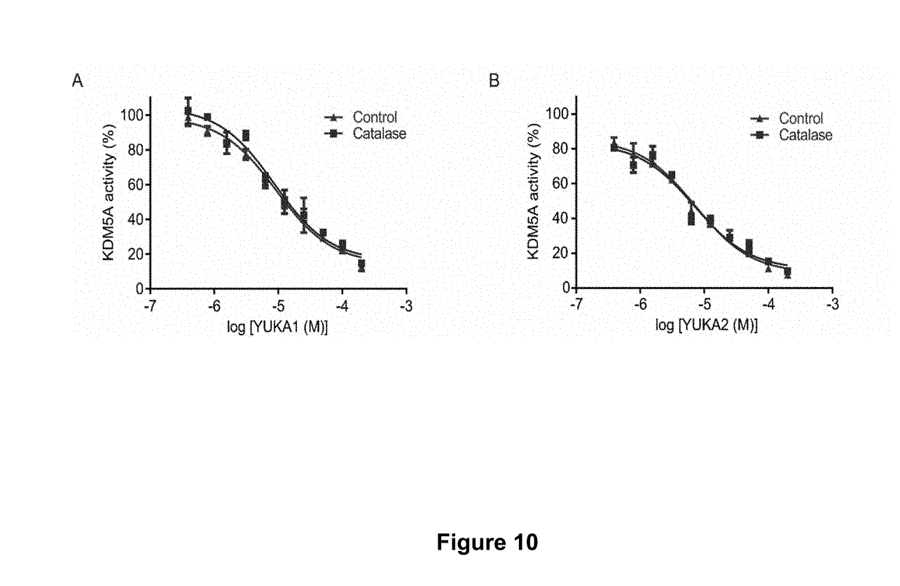

A crystal structure of truncated KDM5A was recently solved (Horton et al., 2016, J. Biol. Chem 291:2631-2646). A related compound was reported to be a pan-assay interference compound (Baell and Holloway, 2010, J. Med. Chem. 53:2719-2740). YUKA1 and YUKA2 displayed remarkable specificity against KDM5A's closest relatives in the experiments presented here and they proved to be extremely useful tool compounds. Furthermore, catalase, a hydrogen peroxide scavenger, did not affect the inhibitory potency of YUKA1 and YUKA2 against KDM5A, indicating that hydroxyl radicals formed by potential Fenton chemical reactions are not inactivating the protein (FIG. 10). Efforts to further characterize the mode of action of these compounds are ongoing.

The tool compounds described here allowed determination of the necessity of KDM5A's catalytic activity in two of those settings. YUKA1 inhibited proliferation of HeLa cervical cancer cells and ZR-75-1 breast cancer cells (FIGS. 5B and 6B). Although not wishing to be bound by any particular theory, these results suggest that the demethylase activity of KDM5A is indeed required for the development of drug tolerance to two different targeted therapies, a small molecule and a monoclonal antibody targeting members of the epidermal growth factor receptor family in lung and breast cancer, respectively (FIG. 8). This data provides rationale to consider use of KDM5A inhibitors to sensitize cells to established anti-cancer therapy regimens. Taken together, this screen identified specific inhibitors that can be used to study the biology of the KDM5A demethylase.

### Example 2

### Examples of Found Inhibitors of KDM5A from High Throughput Screening, (IC₅₀ in μM)

| | | KDM5A | |
|---|---|---|---|
| | Compound | IC50 | |
| Structure | ID | (μm) | Name |
| 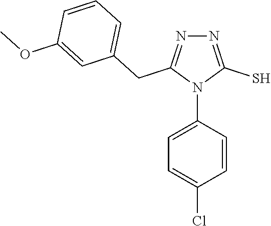 [SMILES: COc1cccc(Cc2nnc(S)n2-c2ccc(Cl)cc2)c1] | YU017175 | 0.057 | 4-(4-chlorophenyl)-5-(3- methoxybenzyl)-4H-1,2,4- triazole-3-thiol |
| 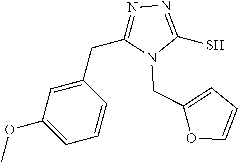 [SMILES: COc1cccc(Cc2nnc(S)n2Cc2ccco2)c1] | YU017176 | 1.76 | 4-(furan-2-ylmethyl)-5-(3- methoxybenzyl)- 4H-1,2,4-triazole-3-thiol |
| 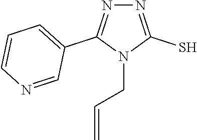 | YU125526 | 0.197 | 4-allyl-5-(pyridin-3-yl)-4H- 1,2,4-triazole-3-thiol |
| 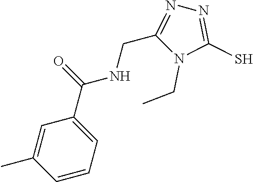 [SMILES: CCn1c(S)nnc1CNC(=O)c1cccc(C)c1] | YU126648 | 0.159 | N-((4-ethyl-5-mercapto-4H- 1,2,4-triazol-3-yl)methyl)-3- methylbenzamide |
| 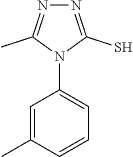 | YU128402 | 0.079 | 5-methyl-4-(m-tolyl)-4H- 1,2,4-triazole-3-thiol |
| 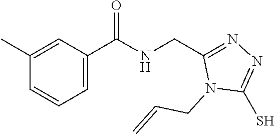 [SMILES: C=CCn1c(S)nnc1CNC(=O)c1cccc(C)c1] | YUKA2; YU149014; Chembridge 7840569 YUKA2 | 2.66 | N-((4-allyl-5-mercapto-4H- 1,2,4-triazol-3-yl)methyl)-3- methylbenzamide |
| 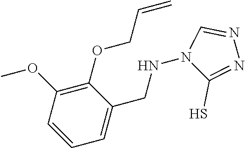 [SMILES: C=CCOc1c(CNn2cnnc2S)cccc1OC] | YUKA1; YU149411; Chembridge 7870547; YUKA1 | 1.32 | 4-((2-(allyloxy)-3- methoxybenzyl)amino)-4H- l,2,4-triazole-3-thiol |
| 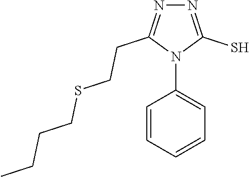 [SMILES: CCCCSCCc1nnc(S)n1-c1ccccc1] | YU149596; Chembridge 7918866; Compound 4 | 0.169 | 5-(2-(butylthio)ethyl)-4- phenyl-4H-1,2,4-triazole-3- thiol |
| 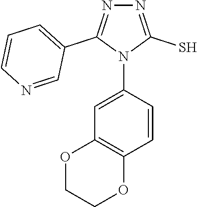 [SMILES: Sc1nnc(-c2cccnc2)n1-c1ccc2c(c1)OCCO2] | YU151512 | 0.367 | 4-(2,3- dihydrobenzo[b][1,4]dioxin- 6-yl)-5-(pyridin-4-yl)-4H- 1,2,4-triazole-3-thiol |

### Example 3

### YUKA1(KDM5Ai), but not KDM5-C70 (pan-KDM5i), Suppresses Growth of HeLa and MDA-MB231 Triple Negative Breast Cancer Cells

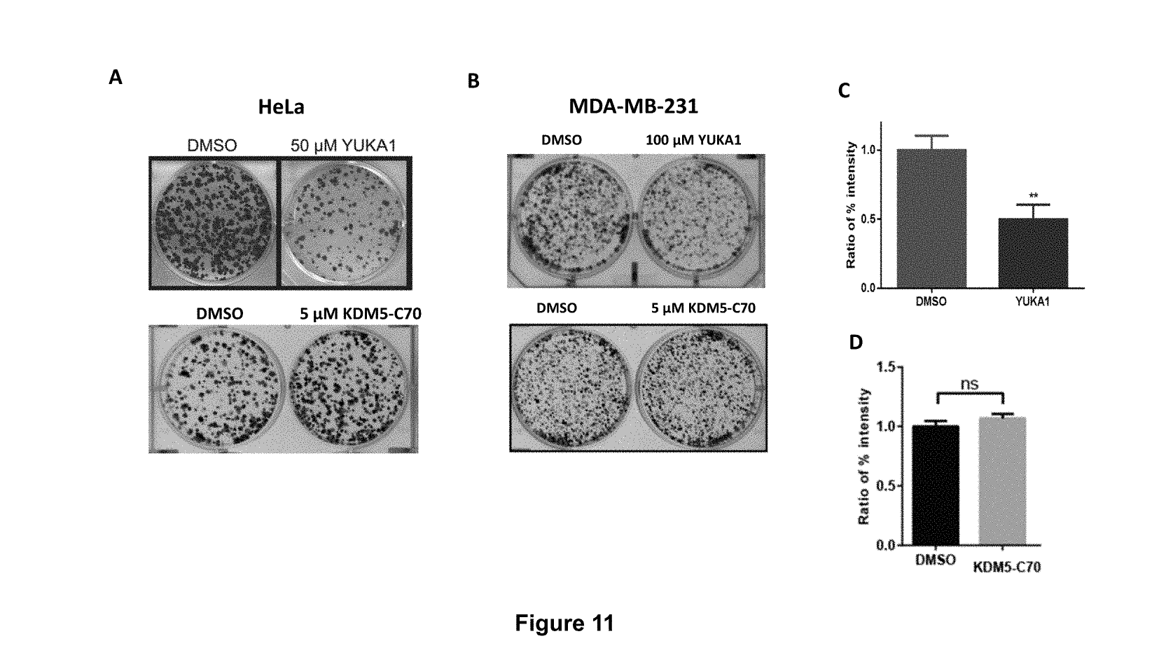

Experimental data demonstrated that YUKA1(KDM5Ai), but not KDMS-C70 (pan-KDM5i), suppresses growth of HeLa and MDA-MB231 triple negative breast cancer cells. HeLa (FIG. 11A) and MDA-MB231 cells (FIG. 11B) were treated with DMSO, 5 μM KDMS-C70 or 50 or 100 μM YUKA1 in colony-formation assays. The tables in FIGS. 11C and 11D depict the quantification of experimental data.

1. A pharmaceutical composition comprising a compound, or a salt or solvate thereof, selected from the group consisting of a compound of formulae (I)-(IV): 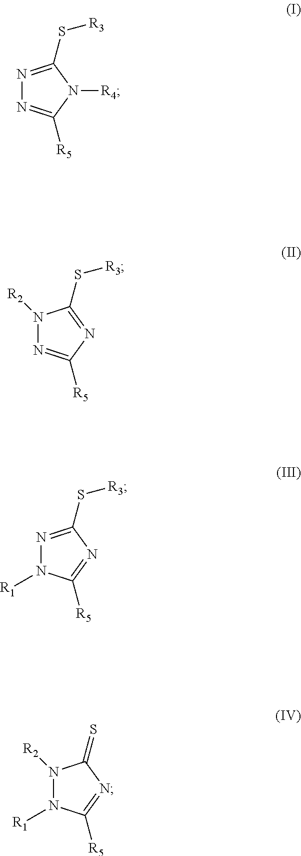
wherein in formulae (I)-(IV):
R¹, R², and R⁵ are each independently selected from the group consisting of H, C₁-C₆ alkyl, aryl-(C₁-C₃)alkyl, substituted aryl-(C₁-C₃)alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, and substituted heteroaryl;
R³ is selected from the group consisting of H, —C(O)R⁶, and —SR⁸;
R⁴ is selected from the group consisting of H, C₁-C₆ alkyl, substituted C₁-C₆ alkyl, C₂-C₆ alkenyl, aryl-(C₁-C₃)alkyl, substituted aryl-(C₁-C₃)alkyl, heteroaryl-(C₁-C₃)alkyl, substituted heteroaryl-(C₁-C₃)alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, and —NHR⁷;
R⁶ is selected from the group consisting of C₁-C₆ alkyl, aryl, and heteroaryl;
R⁷ is selected from the group consisting of H, C₁-C₆ alkyl, substituted C₁-C₆ alkyl, aryl, substituted aryl, aryl-(C₁-C₃)alkyl, substituted aryl-(C₁-C₃)alkyl, heteroaryl, substituted heteroaryl, heterocyclyl, substituted heterocyclyl, —C(O)R⁹, —S(O)R⁹, —S(O)₂R⁹;
R⁸ is selected from the group consisting of H, C₁-C₆ alkyl, substituted C₁-C₆ alkyl, C₂-C₆ alkenyl, C₃-C₇ cycloalkyl, substituted C₃-C₇ cycloalkyl, aryl, substituted aryl, heterocyclyl, substituted heterocyclyl, heteroaryl, and substituted heteroaryl; and
R⁹ is selected from the group consisting of C₁-C₆ alkyl, substituted C₁-C₆ alkyl, C₃-C₇ cycloalkyl, substituted C₃-C₇ cycloalkyl, aryl, substituted aryl, heterocyclyl, substituted heterocyclyl, heteroaryl, and substituted heteroaryl.

2. The composition of claim 1, wherein the compound of formulae (I)-(IV), is selected from the group consisting of a compound of formulae (Ia), (IIa), (IIIa), and (Ib), or a salt or solvate thereof:

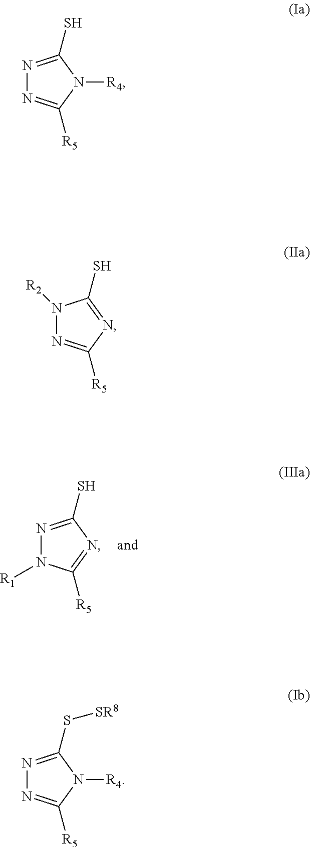

3.-7. (canceled)

8. The composition of claim 1, wherein R⁴ is selected from the group consisting of H, C₁-C₆ alkyl, C₂-C₆ alkenyl, aryl, substituted aryl, heterocyclyl, aryl-(C₁-C₃)alkyl, heteroaryl-(C₁-C₃)alkyl, and —NHR⁷, wherein R⁷ is substituted aryl-(C₁-C₃)alkyl.

9. The composition of claim 1, wherein R⁵ is selected from the group consisting of H, C₁-C₆ alkyl, substituted C₁-C₆ alkyl, aryl, substituted aryl-(C₁-C₃)alkyl, substituted aryl, and heteroaryl.

10. (canceled)

11. The composition of claim 1, wherein the compound is selected from the group consisting of:

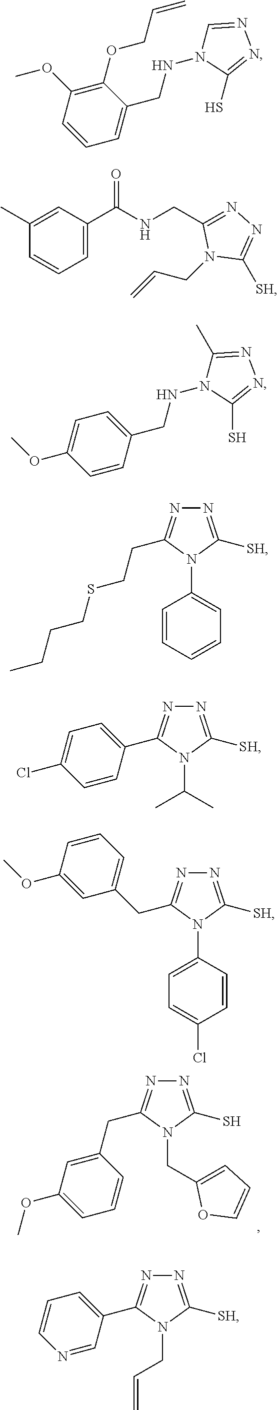

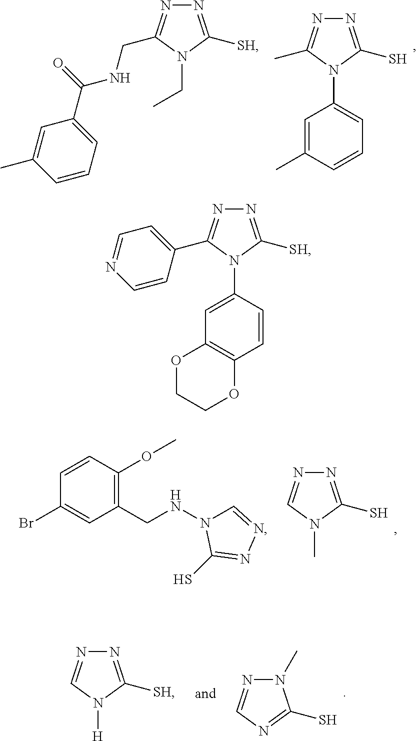

12. The composition of claim 1, wherein the compound is selected from the group consisting of

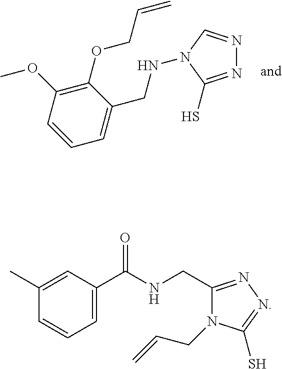 [SMILES: C=CCOc1c(CNn2cnnc2S)cccc1OC ; C=CCn1c(S)nnc1CNC(=O)c1cccc(C)c1]

13. A method of treating or preventing cancer in a subject in need thereof, the method comprising administering to the subject a therapeutically effective amount of a pharmaceutical composition comprising a compound selected from the group consisting of a compound of formulae (I)-(IV): 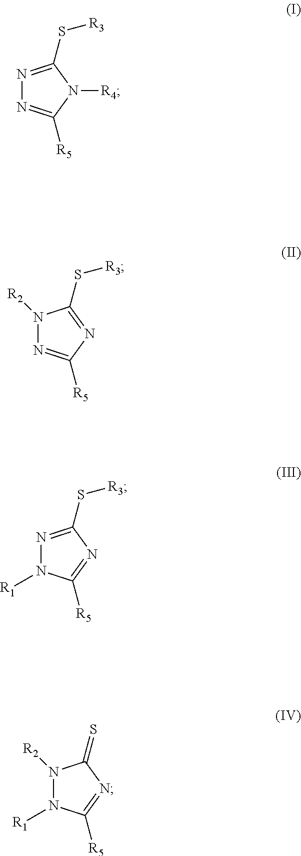
wherein in formulae (I)-(IV):
R¹, R², and R⁵ are each independently selected from the group consisting of H, C₁-C₆ alkyl, aryl-(C₁-C₃)alkyl, substituted aryl-(C₁-C₃)alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, and substituted heteroaryl;
R³ is selected from the group consisting of H, —C(O)R⁶, and —SR⁸;
R⁴ is selected from the group consisting of H, C₁-C₆ alkyl, substituted C₁-C₆ alkyl, C₂-C₆ alkenyl, aryl-(C₁-C₃)alkyl, substituted aryl-(C₁-C₃)alkyl, heteroaryl-(C₁-C₃)alkyl, substituted heteroaryl-(C₁-C₃)alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, and —NHR⁷;
R⁶ is selected from the group consisting of C₁-C₆ alkyl, aryl, and heteroaryl;
R⁷ is selected from the group consisting of H, C₁-C₆ alkyl, substituted C₁-C₆ alkyl, aryl, substituted aryl, aryl-(C₁-C₃)alkyl, substituted aryl-(C₁-C₃)alkyl, heteroaryl, substituted heteroaryl, heterocyclyl, substituted heterocyclyl, —C(O)R⁹, —S(O)R⁹, —S(O)₂R⁹;
R⁸ is selected from the group consisting of H, C₁-C₆ alkyl, substituted C₁-C₆ alkyl, C₂-C₆ alkenyl, C₃-C₇ cycloalkyl, substituted C₃-C₇ cycloalkyl, aryl, substituted aryl, heterocyclyl, substituted heterocyclyl, heteroaryl, and substituted heteroaryl; and
R⁹ is selected from the group consisting of C₁-C₆ alkyl, substituted C₁-C₆ alkyl, C₃-C₇ cycloalkyl, substituted C₃-C₇ cycloalkyl, aryl, substituted aryl, heterocyclyl, substituted heterocyclyl, heteroaryl, and substituted heteroaryl.

14. The method of claim 13, wherein the compound is selected from the group consisting of

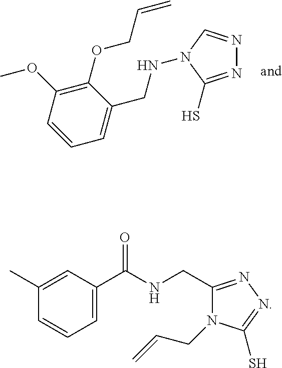 [SMILES: C=CCOc1c(CNn2cnnc2S)cccc1OC ; C=CCn1c(S)nnc1CNC(=O)c1cccc(C)c1]

15. The method of claim 13, wherein administration of the pharmaceutical composition to the subject inhibits the activity of at least one JARID1 demethylase in the subject.

16. (canceled)

17. The method of claim 13, wherein the cancer comprises a solid cancer selected from the group consisting of breast cancer, prostate cancer, melanoma, lung cancer, gastric cancer, hepatocellular cancer, glioblastoma, neuroendocrine cancers, pancreatic cancer, and any combinations thereof.

18. (canceled)

19. The method of claim 18, wherein the breast cancer comprises at least one HER2-positive breast cancer cell resistant to trastuzumab.

20. (canceled)

21. The method of claim 18, wherein the lung cancer comprises at least one EGFR-mutant lung cancer cell resistant to gefitinib.

22. (canceled)

23. The method of claim 14, wherein the subject is further administered an additional compound selected from the group consisting of a chemotherapeutic agent, an anti-cell proliferation agent, and any combinations thereof.

24. The method of claim 23, wherein the chemotherapeutic agent comprises an alkylating agent, nitrosourea, antimetabolite, antitumor antibiotic, plant alkyloid, taxane, hormonal agent, bleomycin, hydroxyurea, L-asparaginase, or procarbazine.

25. The method of claim 24, wherein the anti-cell proliferation agent comprises granzyme, a Bcl-2 family member, cytochrome C, or a caspase.

26. (canceled)

27. The method of claim 23, wherein the pharmaceutical composition and the additional compound are co-formulated and co-administered to the subject.

28.-30. (canceled)

31. A kit comprising an applicator, an instructional material for use thereof, and a compound selected from the group a compound selected from the group consisting of a compound of formulae (I)-(IV): 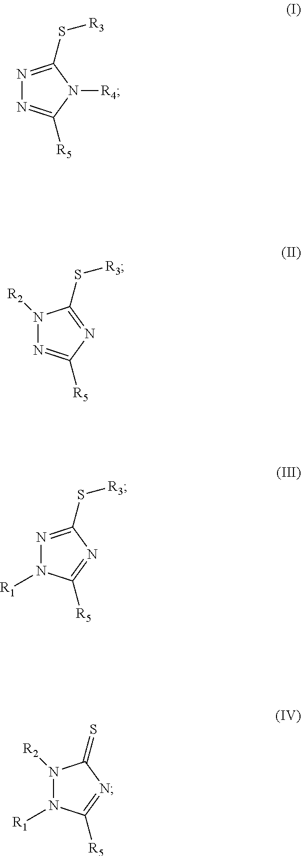
wherein in formulae (I)-(IV):
R¹, R², and R⁵ are each independently selected from the group consisting of H, C₁-C₆ alkyl, aryl-(C₁-C₃)alkyl, substituted aryl-(C₁-C₃)alkyl, substituted C₁-C₆ alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, and substituted heteroaryl;
R³ is selected from the group consisting of H, —C(O)R⁶, and —SR⁸;
R⁴ is selected from the group consisting of H, C₁-C₆ alkyl, substituted C₁-C₆ alkyl, C₂-C₆ alkenyl, aryl-(C₁-C₃)alkyl, substituted aryl-(C₁-C₃)alkyl, heteroaryl-(C₁-C₃)alkyl, substituted heteroaryl-(C₁-C₃)alkyl, C₁-C₆ haloalkyl, C₃-C₇ cycloalkyl, substituted C₃-C₇ cycloalkyl, aryl, substituted aryl, heterocyclyl, substituted heterocyclyl, heteroaryl, substituted heteroaryl, and —NHR⁷;
R⁶ is selected from the group consisting of C₁-C₆ alkyl, aryl, and heteroaryl;
R⁷ is selected from the group consisting of H, C₁-C₆ alkyl, substituted C₁-C₆ alkyl, aryl, substituted aryl, aryl-(C₁-C₃)alkyl, substituted aryl-(C₁-C₃)alkyl, heteroaryl, substituted heteroaryl, heterocyclyl, substituted heterocyclyl, —C(O)R⁹, —S(O)R⁹, —S(O)₂R⁹;
R⁸ is selected from the group consisting of H, C₁-C₆ alkyl, substituted C₁-C₆ alkyl, C₂-C₆ alkenyl, C₃-C₇ cycloalkyl, substituted C₃-C₇ cycloalkyl, aryl, substituted aryl, heterocyclyl, substituted heterocyclyl, heteroaryl, and substituted heteroaryl; and
R⁹ is selected from the group consisting of C₁-C₆ alkyl, substituted C₁-C₆ alkyl, C₃-C₇ cycloalkyl, substituted C₃-C₇ cycloalkyl, aryl, substituted aryl, heterocyclyl, substituted heterocyclyl, heteroaryl, and substituted heteroaryl.
wherein the instructional material comprises instructions for preventing or treating cancer in a subject;
wherein the instructional material recites that the subject is administered a therapeutically effective amount of a pharmaceutical composition comprising the compound contained in the kit, whereby the cancer in the subject is treated or prevented.

32. The kit in claim 31, wherein the cancer comprises breast cancer, prostate cancer, melanoma, lung cancer, gastric cancer, hepatocellular cancer, glioblastoma, and any combinations thereof.

33. The kit of claim 32, wherein the breast cancer comprises at least one HER2-positive breast cancer cell resistant to trastuzumab.

34. (canceled)

35. The kit in claim 32, wherein the lung cancer comprises at least one EGFR-mutant lung cancer cell resistant to gefitinib.

36. (canceled)
